(19) United States
(12) Patent Application Publication
(10) Pub. No.: US 2023/0130823 A1
(43) Pub. Date: Apr. 27, 2023

(54) PROCESS FOR THE PRODUCTION OF ANTITUMOUR PHARMACEUTICAL COMPOSITIONS USING PUSH-PULL BUTADIENES, COMPOUNDS AND USES THEREOF

(71) Applicants: ANHANGUERA EDUCACIONAL PARTICIPAÇÕES S/A, Valinhos (BR); FUNDAÇÃO UNIVERSIDADE FEDERAL DO ABC, Santo André (BR); INSTITUTO BUTANTAN, São Paulo (BR)

(72) Inventors: Anderson ORZARI RIBEIRO, Santo André (BR); Carolina PASSARELLI GONÇALVES, São Paulo (BR); Durvanei AUGUSTO MARIA, São Paulo (BR); José Agustín QUINCOCES SUÁREZ, São PAulo (BR); Manuel SALUSTIANO ALMEIDA SAAVEDRA, São Paulo (BR)

(73) Assignees: ANHANGUERA EDUCACIONAL PARTICIPAÇÕES S/A, Valinhos (BR); FUNDAÇÃO UNIVERSIDADE FEDERAL DO ABC, Santo André (BR); INSTITUTO BUTANTAN, São Paulo (BR)

(21) Appl. No.: 17/771,124

(22) PCT Filed: Oct. 23, 2020
(86) PCT No.: PCT/BR2020/050437

§ 371 Date: Apr. 22, 2022

(30) Foreign Application Priority Data
Oct. 25, 2019 (BR) BR10 2019 0225297

(51) Int. Cl. C07C 319/14; C07C 323/27; C07C 319/28; C07C 253/30; C07C 255/38; C07C 253/34; C07C 201/12; C07C 201/16; C07C 205/41; C07C 67/40; C07C 67/58; C07C 69/94; C07C 45/45; C07C 45/79; C07C 49/235; C07C 255/35; C07C 205/09; C07C 255/40; C07C 49/255

(52) U.S. Cl. CPC C07C 319/14; C07C 323/27; C07C 319/28; C07C 253/30; C07C 255/38; C07C 253/34; C07C 201/12; C07C 201/16; C07C 205/41; C07C 67/40; C07C 67/58; C07C 69/94; C07C 45/45; C07C 45/79; C07C 49/235; C07C 255/35; C07C 205/09; C07C 255/40; C07C 49/255

(57) ABSTRACT

The present invention reports the obtaining of carbonyl compounds and derivatives, through syntheses with high yield and purity, providing anti-humoral active principles with selective antiproliferative properties and anti-metastatic activity.

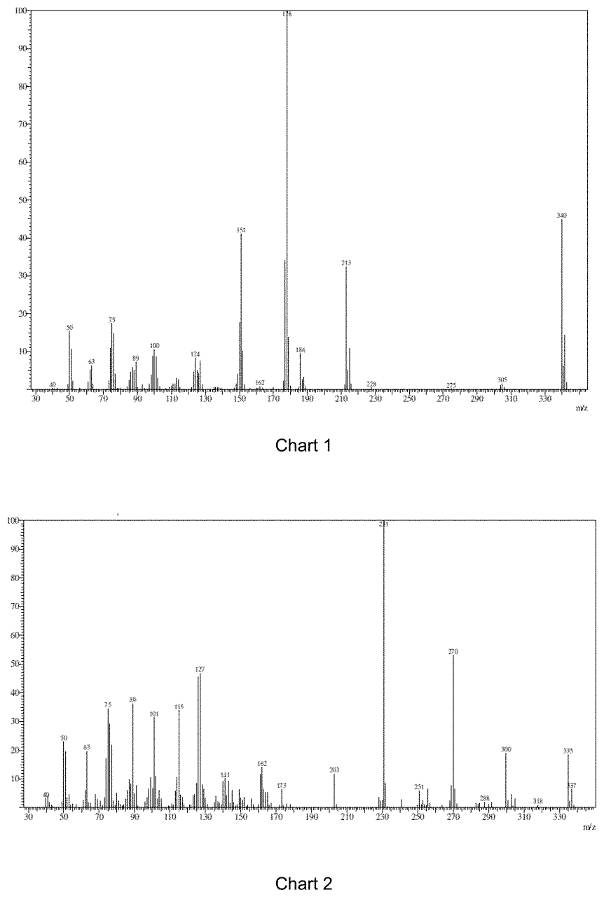

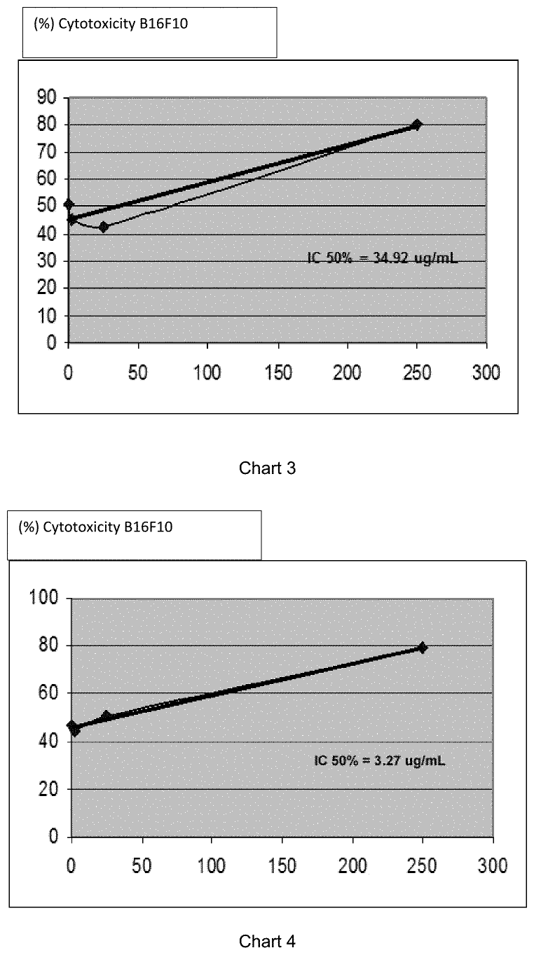

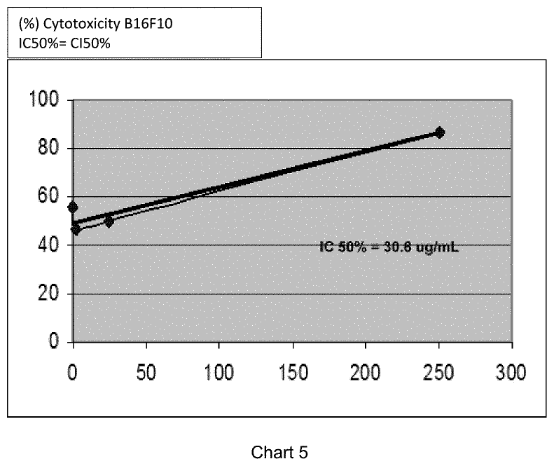

The present invention refers to the development of new polyfunctional push-pull butadienes and their O and C-prenylated, benzoylated and iodide derivatives, with high electronic conjugation in the lateral chain. These compounds exhibit high anti-tumor selectivity, causing cell death by apoptosis, also show anti-metastatic and non-mutagenic properties in the experimental studies performed.

### CROSS-REFERENCE TO RELATED APPLICATIONS

This application is a 35 U.S.C. § 371 National Phase Entry Application of International Application No. PCT/BR2020/050437 filed Oct. 23, 2020, which designates the U.S. and claims benefit of BR Application No. BR 1020190225297 filed Oct. 25, 2019, the contents of both of which are incorporated herein by reference in their entireties.

The present invention reports the obtaining of carbonyl compounds and derivatives, through syntheses with high yield and purity, providing anti-humoral active principles with selective antiproliferative properties and anti-metastatic activity.

### THE PRINCIPLES OF THE INVENTION

Cancer disease covers every day more victims worldwide in all age groups. The current pharmaceutical market also faces the increasing resistance of cancer cells to conventional chemotherapeutic treatments, which have high toxicity. There are also no new, more effective selective active principles on the market, which also have other important properties, such as causing cancer cells to die from apoptosis and preventing them from migrating to other tissues and organs (antimetastatic effect), as well as not causing secondary tumors formation non-mutagenic effect).

The present invention refers to the development of new polyfunctional push-pull butadienes and their O and C-prenylated, benzoylated and iodide derivatives, with high electronic conjugation in the lateral chain. These compounds exhibit high anti-tumor selectivity, causing cell death by apoptosis, also show anti-metastatic and non-mutagenic properties in the experimental studies performed.

Nowadays, we see important advances in the fight against cancer, but even with some successes, the perspectives are not encouraging. It is estimated that in the year 2030, 12 million cancer people in the world will die from the increase and aging of the population. The problem is further aggravated by the failure of antineoplastic chemotherapy due to drugs resistance.

Since the onset of chemotherapy for cancer, many of the ways in which cancer cells “escape” from the chemical agent have been identified. At the moment cells develop resistance to a drug, they may also develop cross-resistance to other drugs, chemically mechanistically unrelated, in a phenomenon known as multi-drug resistance (MDR).

The development of new active principles with antitumor properties is still essential to try to reduce the number of patients who are victims of this terrible disease.

### STATE OF THE ART

No information related to the antitumor properties of O and C-prenylated compounds, benzoylated derived from push-pull butadienes with high electronic conjugation in the lateral chain, was found in the bibliographic review Sci Finder, or in the specialized patent sites.

Gompper (Gompper and Seybold, Angew. Chem. 80, 804, 1968) was the first to study the special reactive behavior of olefins activated by the influence of donor and electron-withdrawing organic functional groups, located on opposite sides of a double C═C binding, giving the name to these compounds of “push-pull alkenes”.

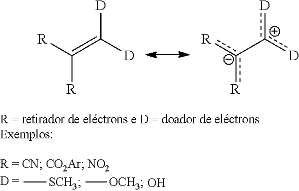

This neighboring arrangement of push-pull substituents causes considerable polarization in the molecules, which conditions the special properties of this group of substances. In contrast to classical alkenes that react with electrophilic reagents, with loss of double C═C binding, they appreciate the “push-pull alkenes” preference for reactions with nucleophiles, replacing an electron donating group and maintaining the unsaturated system.

The push-pull pathway has already been used to obtain drugs of recognized biological importance, such as 5-chloro-4-hydroxy-2-(1H)-pyridone, dihydropyrimidine dehydrogenase inhibitor (YANO, 1993), 5-(chloromethyl)-furo[2,3-b]pyridine, HIV protease inhibitor (BHUTATHY, 1995), 4-hydroxy-niconitic acid, which has antibacterial and anti-rheumatic activity (MITTELBACH, 1985) and the compound N-(2-[5-(dimethylaminomethyl)furan-2-il)methyltio]ethyl)-N-methyl-2-nitroetene-1,1-diamine, H2 receptor antagonist, also known as ranitidine (NAVARRO, 1995).

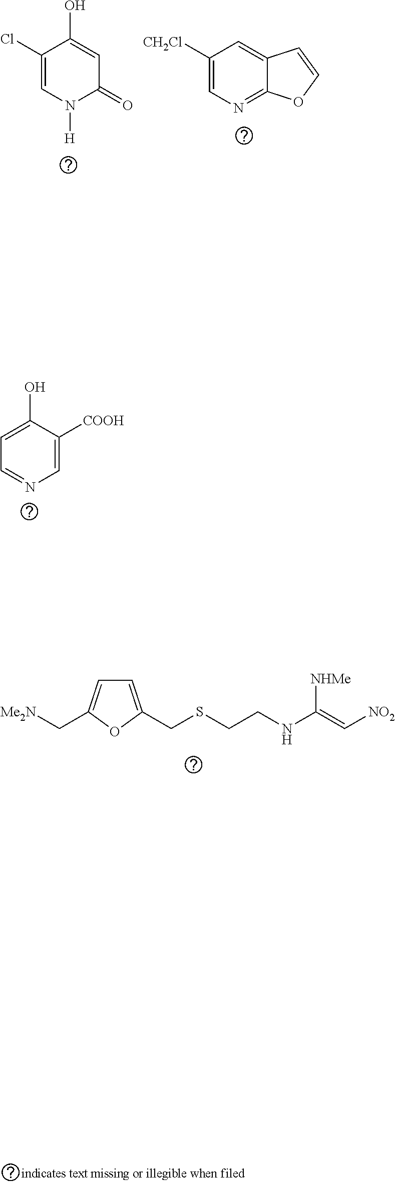

The synthesis of 5-chloro-4-hydroxy-2-(1H)-pyridone, for example, had already been reported previously without the use of the push-pull pathway, but required several steps of synthesis and the final product was obtained with yields of between 12 and 26%. In 1993 Yano (YANO, 1993) reported the synthesis of this same substance through the use of the push-pull pathway with 91% yield and four synthesis steps.

The 4-hydroxy-nicotinic acid is of great importance as a component of penicillins and cephalosporins. It is proven that 4-hydroxy-nicotinic acid also has an anti-rheumatic property and also has an anti-inflammatory power superior to that of salicylic acid. Several authors had already reported the synthesis of this product, but the yields obtained were low. In 1985, the synthesis of 4-hydroxy-nicotinic acid was described with the use of the push-pull pathway, obtaining 75% of yield by using the push-pull pathway in only three synthesis steps (MITTELBACH, 1985).

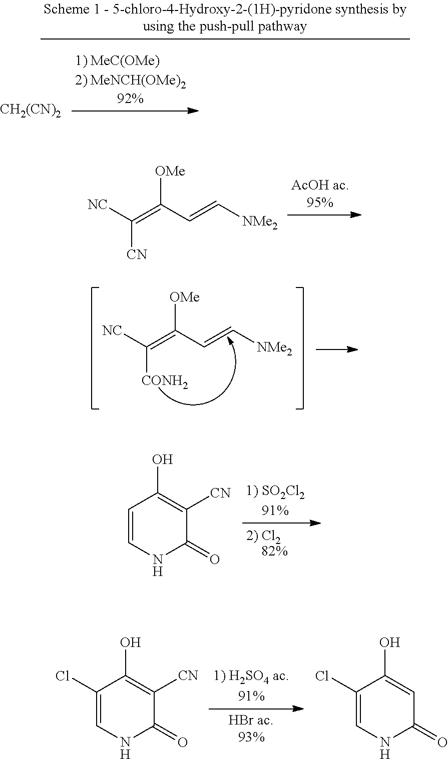

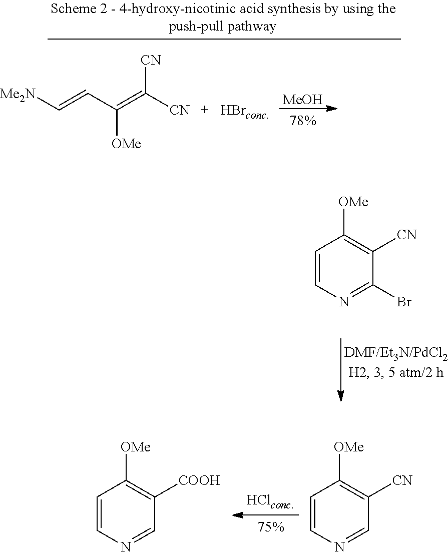

The compound 4-hydroxy-pyrazolo [3,4-d] pyrimidine, allopurinol, can also be highlighted. Important antiarthritic, which presents antitumor and antileukemic activity, which was also obtained through the use of the push-pull pathway (TOMINAGA, 1990).

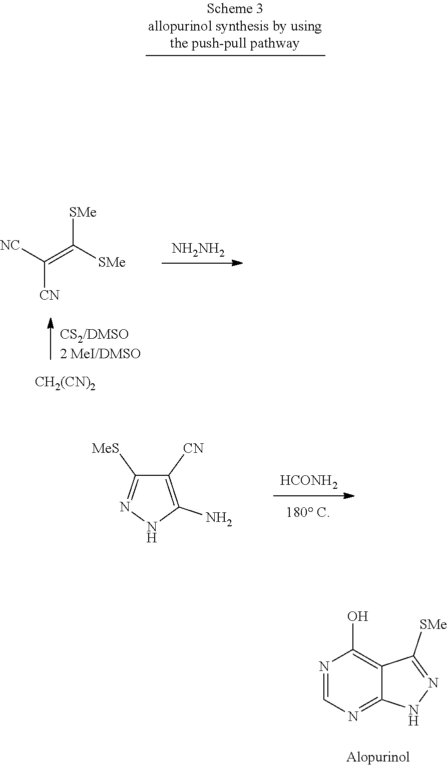 [SMILES: CSC(SC)=C(C#N)C#N ; "NH2NH2" ; "CH2(CN)2" ; CSc1n[nH]c(N)c1C#N ; "HCONH2" ; CSc1n[nH]c2ncnc(O)c12]

There is a considerable number of acyclic and cyclic push-pull systems reported in the literature. Among them are chlorovinyl aldehyde and push-pull butadienes.

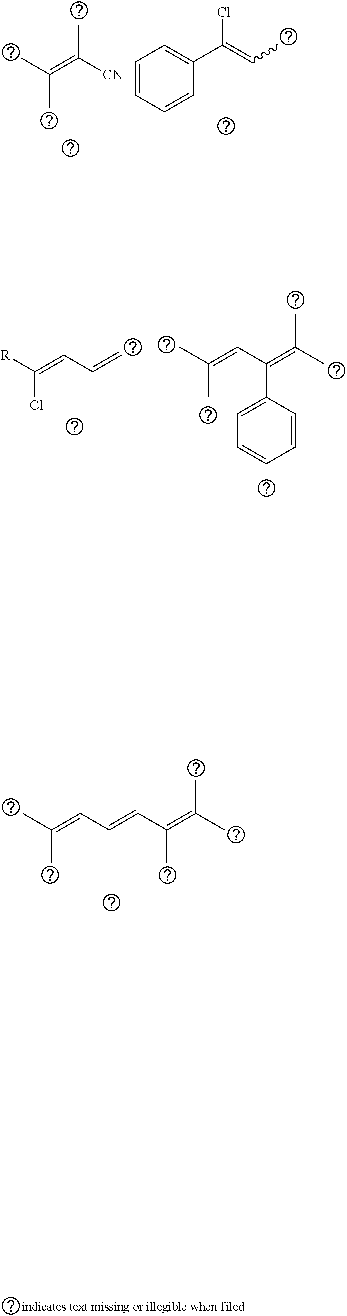

Push-pull butadienes are “olefins” formed by four carbonates with two double interleaved bonds, which present electron donating groups (MeS, among others) at one end of the molecule and withdrawing groups (CN, among others) at another end. These conjugated push-pull systems can be obtained by Knoevenagel condensation from chlorovinyl aldehyde or Liebscher salts with CH-acid compounds, which eliminate a water molecule to form an α,β-unsaturated compound.

### DETAILED AND TECHNICAL DESCRIPTION OF THE INVENTION

### 01) Obtaining 4-(3-methylbut-2-enyl-oxyi)acetophenone

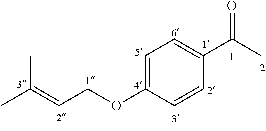

To obtain this product, a nucleophilic substitution reaction was made from 0.01 mol (1.36 g) of 4-hydroxy-acetophenone, 10 mL of anhydrous dimethylformamide and 0.03 mol (4.14 g) of anhydrous potassium carbonate at a constant temperature of 40° C. Next, 0.02 mol (2.98 g; 2.32 mL) of prenyl bromide is added, drop by drop, and in the presence of argon to eliminate possible oxygen interference. The reaction time is 8 hours. After this period, the mixture is poured into water with ice, where the precipitation of the product will occur as a white solid. It is filtered and dried at room temperature. The product is obtained with a high degree of purity. Yield: 80%. Melting point: 47° C. ¹H-RMN (DMSO-d6): δ=7.91 (d, 2H, H-2′/H-6′), 6.92 (d, 2H, H-3′/H-5′), 5.50 (m, 1H, H-2″), 4.61 (d, 2H, H-1″), 2.61 (s, 3H, CH3CO), 1.82 (s, 3H, CH3 prenyl), 1.70 (s, 3H, CH3 prenyl). ¹³C-RMN (CDCl₃): δ=196.7 (C-1), 162.71 (C-4′), 138.8 (C-3″), 130.5 (C-2′/C-6′), 130.1 (C-1′), 118.8 (C-2″), 114.2 (C-5′/C-3′), 64.9 (C-1″), 26.2 (C-2), 25.7 (CH₃-prenyl), 18.1 (CH₃-prenyl). IV (film): ν=3029 (═CH), 2970 (CH₃), 2875 (CH₂), 1690 (C═O), 1242 (C—O) cm⁻¹. MS (70 eV): m/z=204 M⁺, 189 (M⁺- 15 (CH₃); 161 M⁺-43 (CH₃CO); 69 (prenyl), 43 (CH₃CO). Quantitative elementary analysis: C₁₃H₁₆O₂ (204,26): calculated: C 76.44; 7.89 H; found: C 76.40; H 7.84

### 02) Obtaining 2-Cyano-3-methyl-3-[4-(3-methylbut-2-enoyl-oxy)-phenyl]acrylonitrile

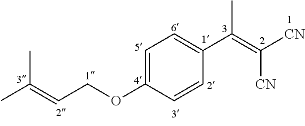 [SMILES: CC(C)=CCOc1ccc(C(C)=C(C#N)C#N)cc1]

This compound is obtained using the Knoevenagel condensation technique. Starting with 0.01 mol (2.04 g) of 4-(3-methylbut-2-enyloxy)acetophenone, 9.5 mL of acetic acid, 0.01 mol (0.66 g) of malonitrile, 3.5 g of ammonium acetate and 40 mL of toluene are added. This mixture is placed under reflux with Dean Stark at a temperature between 120-140° C. for 8 hours. The solvent is rotaevaporated obtaining a brown solid, which is extracted with ethyl ether and washed several times with water. Yield: 70%. Melting point: 71° C. ¹H-RMN (DMSO-d₆): δ=7.59 (d, 2H, H-2′/H-6′), 6.99 (d, 2H, H-3′/H-5′), 5.42 (m, 1H, H-2″), 4.58 (d, 2H, H-1 ″), 2.58 (s, 3H, CH₃(C═C(CN)₂)), 1.78 (s, 3H, CH₃-prenyl), 1.70 (s, 3H, CH₃-prenyl). ¹³C-RMN (DMSO-d₆): δ=173.792 (C-3), 162.430 (C-4′), 139.158 (C-3′), 129.720 (C-2′/6′), 118.623 (C-2′″), 115.014 (C-3′/C-5′), 113.612 (CN), 113,350 (CN), 81.658 (C-2), 65.118 (C-1″), 25,715 (CH₃-prenyl); 23.674 (CH₃—C═C(CN)₂), 18.164 (CH₃-prenyl). DEPT (DMSO-d₆): δ=129.736 (C-2′/C-6′); 118.632 (C-2″); 115.026 (C-3′/C-5′); 65.129 (C-1″); 25.735 (CH₃-prenyl); 23.695 (CH₃—C═C(CN)₂), 18.183 (CH₃-prenyl). IV (film): ν=3026 (═C—H), 2975 (CH₃), 2877 (CH₂), 2220 (CN), 1248 (C—O) cm⁻¹. MS (70 EV): M/z=252 M, 226 (M−26), 69 (prenyl group). Quantitative elementary analysis: C₁₆H₁₆N₂O (252,31): calculated: C 76.16, H 6.39, N 11.11; found: C 76.13, H 6.41, N 11.15.

### 03) Obtaining 4,4-Bis-ethylsulfanyl-2-[4-3-methylbut-2-enyloxy)-phenyl]-buta-1,3-dien-1,1-dicarbonitrile

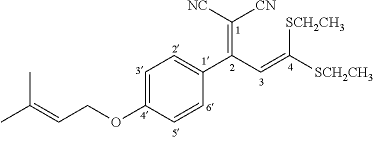

A mixture composed of 0.0068 mol of 2-cyan-3-[4-(3-methylbut-2-enyloxy)-phenyl]-but-2-en-nitrile (1.72 g) and 0.0068 mol of carbon disulfide (0.5168 g) in 10 mL absolute dimethylformamide is stirred at room temperature under argon atmosphere. 0.0136 mol of sodium hydride (0.3264 g) are added by stirring under argon atmosphere for eight hours. Subsequently, 0.0136 mol of ethyl iodide (2.12 g) (1097 μL) are added under agitation. After four hours shaking, the reaction mixture is poured into cold water. It is extracted with ether and the organic phase is washed three times with water, drying with anhydrous sodium sulphate.

Solvent is distilled with the aid of vacuum rotoevaporator and the compound is purified by a silica gel chromatographic column, using a ratio 9:1 toluene/ethyl acetate mixture as a mobile phase. Yield: 87%. ¹H NMR (250 MHz, CDCl₃): δ=7.32 (d, 2H, H-2′/H-6′); 6.98 (d, 2H, H-3′/H-5′); 6.38 (s, 1H, H-3); 5.49 (m, 1H, ═CH-prenyl); 4.61 (d, 2H, CH₂-prenila); 3.01 (q, 2H, CH₂S); 2.90 (q, 2H, CH₂S); 1.78 (s, 3H, CH₃-prenyl); 1.75 (s, 3H, CH₃-prenyl); 1.3 (t, 3H, CH₃-etila); 1.2 (t, 3H, CH₃-ethyl). ¹³C NMR (62.9 MHz, CDCl₃): δ=168.02 (C-2); 162.50 (C-4′); 161.53 (C-4); 138.99 (═C(CH₃)₂-prenyl); 131.03 (C-2′/C-6′); 114.91 (C-3′/C-5′); 129.77 (C-1′); 128.44 (C-1); 118.95 (═CH-prenyl); 117.23 (C-3); 114.74 (CN, CN); 65.07 (CH₂-prenyl); 28.81 (CH₂-ethyl); 28.35 (CH₂-ethyl); 25.88(CH₃-prenyl); 18.29 (CH₃-prenyl); 14.56 (CH₃-ethyl); 12.96 (CH₃-ethyl). DEPT (CDCl₃): δ=131.038 (C-2′/C-6′); 115.103 (C-3′/C-5); 118.949 (═CH-prenyl); 117.207 (C-3); 55.077 (CH₂-prenyl); 28.818 (CH₂-ethyl); 28.357 (CH₂-ethyl); 25.883 (CH₃-prenyl); 18.302 (CH₃-prenyl); 14.569 (CH₃-ethyl); 12.971 (CH₃-ethyl).

### 04) Obtaining 2-Acetyl Benzoate of Phenyl

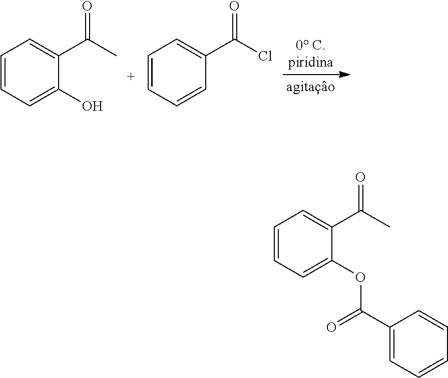

Equimolar quantities of 2-hydroxy-acetophenone and benzoyl chloride in the presence of anhydrous pyridine react at 0° C. under constant agitation to form 2 acetyl benzoate of phenyl. RF of raw material: 0,875 (toluene/ethyl acetate ratio 9/1). RF of the product: 0.75 (toluene/ethyl acetate ratio 9/1). General formula: C₁₅H₁₂O₃. Molecular weight: 240 g/mol

Melting point: 70-74° C. Yield: 97% (2.3 g). Appearance: white solid. IV (KBr): γ=3011 (=aromatic CH), 1737 (C═O, ester), 1686 (C═O, ketone), 1599 (C═C aromatic), 1484 (CH₃ asymmetric deformation), 1361 (CH₃ symmetrical deformation), 707 (probable ortho substitution) cm¹.

### 05) Obtaining 3-Chloro-3-(2-benzoyloxy-phenyl)propenal

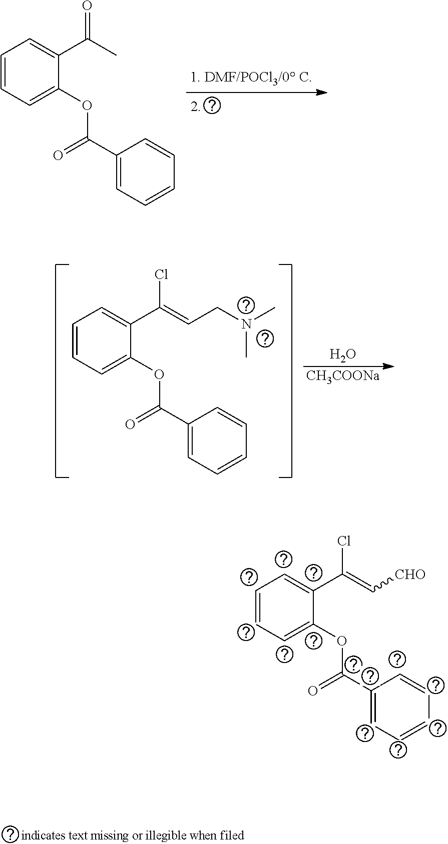

A mixture formed by phosphorus oxychloride and dimethylformamide react at 0° C. Next, it is dripped 2-acetyl benzoate of phenyl dissolved in anhydrous DMF. The reaction mixture is heated to 60° C. for 8 hours. The reaction mixture is placed on a saturated solution of cold sodium acetate, forming chlorovinyl aldehyde. RF of raw material: 0,675 (toluene/ethyl acetate ratio 8/2). RF of the product: 0.875 (toluene/ethyl acetate ratio 8/2). General formula: C₁₆H₁₁O₃Cl. Molecular weight: 286.5 g/mol. Yield: 2.3 g (80%). Appearance: solid light yellow. ¹H NMR (250 MHz, CDCl₃): δ=10.02 (d, 1H, C-1); 9.19 (d, 1H, C-1); aldehyde group signals ratio 1.99/1.00; 8.18 (m, 2H, H-2″/H-6″); multiplet set between ranging from 7.21 to 7.7 (14H, H-3″/H-5″, H-4′, H-5′, H-4′, H-3′, H-2′); 6.41 (D, 2H, H-2). ¹³C NMR (62.9 MHz, CDCl₃): δ=191.169 (C-1); 190.299 (C-1); 164.958 (C-4); 164.831 (C-4); 153.004 (C-3); 148.620 (C-3); 148.238 (C-2′); 148.238 (C-2′); 134.471 (C-4′); 129.152 and 129.103 (C-2″/C-6″); 128.980 (C-3″/C-5″); 126.710 (C-6′); 124.015 (C-2). DEPT (CDCl₃): δ=190.672 (C-1); 189.822 (C-1); 133.948 (C-5″/C-3″); 131.967 (C-6″/C-2′); 131.825 (C-4″); 131.703 (C-4′); 130.692 (C-6′/C-5′); 126.261 (C-3′); 123.469 (C-2).

### 06) Obtaining 2-(1-chloro-4,4-dicyano-buta-1,3-dienyl)phenyl Benzoate

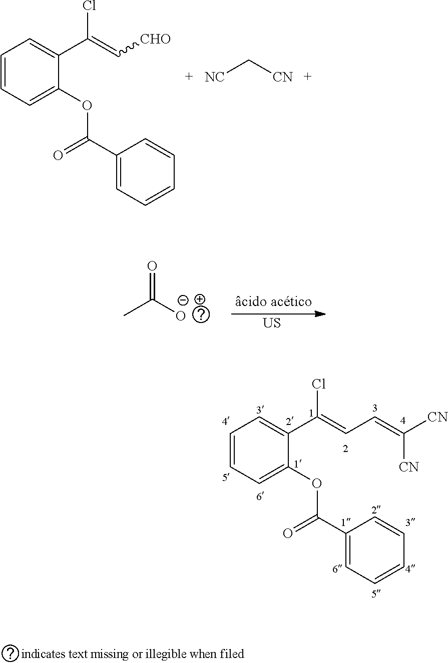

This compound is obtained using the Knoevenagel condensation technique. Starting with 0.004 mol (1.25 g) of 3-chloro-3-(2-benzyloxy-phenyl)propenal, it is added 5 mL of acetic acid, 0.008 mol (0.6 g) of ammonium acetate and 0.04 mol (2.44 g) of malononitrile This mixture is placed under ultrasonic irradiation for 8 hours at room temperature. After this period, the mixture is poured into water, extracted with ethyl acetate, rinsed with distilled water, dried with anhydrous sodium sulphate and the solvent is roto evaporated. RF of raw material: 0.8 (toluene/ethyl acetate ratio 9/1). RF of the product: 0.7 (toluene/ethyl acetate ratio 9/1). General formula: C₁₉H₁₁N₂O₂Cl. Molecular weight: 334.5 g/mol. Yield: 1 g (50%). Appearance: brown solid.

¹H NMR (250 MHz, CDCl₃): δ=8.10 (d, 2H, H-2″/H-6″); 7.80 (d, 2H, H-3″/H-5″); 7.78 (d, 1H, H-3); 6.81 (d, 1H, H-2); aromatic protons seem a set of multiplets ranging from 7.10 to 7.51. ¹³C NMR (62.9 MHz, CDCl₃): δ=155.152 (C-3); 154.638 (C═O); 153.652 (C-1′); 151.471 (C-1); 132.146 (C-4″); 131.972 (C-2″/C-6″); 131.315 (C-3″/C-5″); 130.368 (C-1″); 129.747 (C-5′); 124.694 (C-4′); 124.371 (C-3′); 121.229 (C-6′); 115.960 (C-2); 115.648 (C-2′); 113.217 (CN); 112.155 (CN); 83.163 (C-4). IV (film): ν=3035 (═CH aromatic), 2210 (CN), 1723 (C═O), 1590 (C═C) cm⁻¹.

### 07) Obtaining 2-(1-chloro-4-nitro-buta-1,3-dienyl)phenyl Benzoate

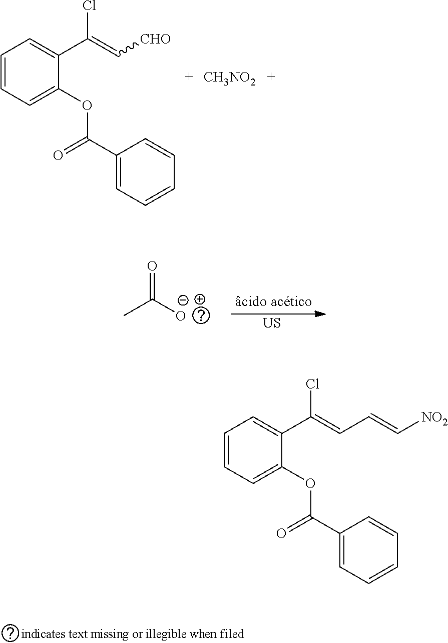

To obtain this product it is mixed 0.004 mol (1.25 g) of 3-chloro-3-(2-benzyloxy-phenyl)propenal (C4), 5 mL of acetic acid, 0.008 mol (0.6 g) of ammonium acetate and 0.04 mol (2.44 g) (2.15 mL) of nitromethane. This mixture is placed under ultrasonic irradiation for 8 hours at room temperature. After this period, the mixture is poured into distilled water, extracted with ethyl acetate, rinsed with distilled water, dried with anhydrous sodium sulphate and the solvent is roto evaporated. For its purification, a chromatographic column filled with silica gel was used, eluting with a mixture of toluene/ethyl acetate (9:1). RF of raw material: 0.7 (toluene/ethyl acetate ratio 9/1). RF of the product: 0.62 (toluene/ethyl acetate ratio 9/1). General formula: C₁₇H₁₂NO₄Cl. Molecular weight: 329.5 g/mol. Yield: 0.5 g (38.5%). Appearance: solid yellow. IV (KBr): γ=3030 (═CH aromatic and aliphatic), 1735 (C═O ester), 1612 and 1592 (C═C aliphatic and aromatic), 1497 and 1341 (NO₂ asymmetric and symmetrical bands respectively), 1252 (C—O, ether), 708 (probable replacement in ortho, vibration of deformation outside the plane) cm⁻¹. E.Weight: M⁺. 329 (1.11%), 294 (M⁺.−35; (1.32%), 105 (PhCO, peak base, (100.0%), 77 (M⁺.−252 (36.65%) phenyl.

### 08) Obtaining 2-(4-acetyl-1-chloro-5-oxo-hexa-1,3-dienyl)phenyl Benzoate

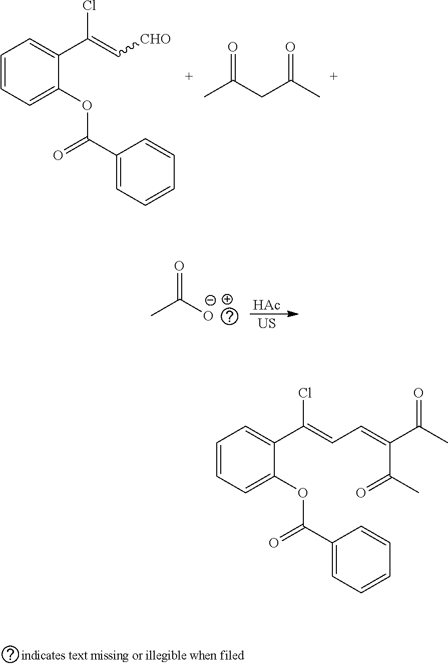

To obtain this product it is mixed 0.007 mol (2 g) of 3-Chloro-3-(2-benzyloxy-phenyl)propenal, 5 mL of acetic acid, 0.014 mol (1.1 g) of ammonium acetate and 0.07 mol (7 g) (7.2 mL) of acetylacetone. This mixture is placed under ultrasonic irradiation for 2 hours at room temperature. After this period, the mixture is poured into distilled water, extracted with ethyl acetate, rinsed with distilled water, dried with anhydrous sodium sulphate and the solvent is roto evaporated. RF of raw material: 0.6 (toluene/ethyl acetate ratio 9/1). RF of the product: 0.4 (toluene/ethyl acetate ratio 9/1). General formula: C₂₁H₁₇O₄Cl. Molecular weight: 368.5 g/mol. Yield: 1.2 g (45%). Appearance: oily orange liquid. E.Weight: M⁺. 369 (25%), 333 (M⁺.−36) 100%, 332 (M⁺. 37)(chloro) 25%, 98 (M⁺.−271) 0.7% C(COCH₃)₂, 105 (1.67%) PhCO group), 164 (192-28) (C—O).

### 09) Obtaining 2-(3-methylbut-2-enyl-oxyi)acetophenone

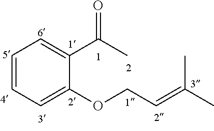

A mixture of o-hydroxy-acetophenone (272 mg, 2 mmol), anhydrous potassium carbonate (828 mg, 6 mmol) and absolute dimethylformamide (10 mL) in inert atmosphere (argon gas) is heated to 40° C., under constant reflux and agitation for 30 minutes. After this period, prenyl bromide (596 mg, 4 mmol) is added drop by drop, and the mixture is allowed for 8 hours under constant agitation and temperature of 40° C. The mixture is poured into distilled water and ice, extracted with ethyl ether three to four times, it is rinsed with NaOH (1 N), also three to four times, to eliminate the residues of o-hydroxy acetophenone without reacting, it is washed with distilled water and anhydrous sodium sulphate is added as a drying agent, it is filtered and the solvent rotaevaporated. The resulting residue is purified using chromatographic column filled with silica gel and toluene/ethyl acetate (9:1) with eluent. Yield: 347 mg (85%); colorless oil. 1H NMR (250 MHz, CDCl3): δ=7.72 (dd, 1H, 3J5′,6′≈7.8 Hz, 4J4′,6′≈2.0 Hz, H-6′), 7.42 (ddd, 1H, 3J3′,4′ 8.5 Hz, 3J4′,5′≈7.5 Hz, 4J4′,6′ 2.0 Hz, H-4′), 6.96 (“dt”, 1H, 4J3′,5′ 1.0 Hz, H-5′), 6.95 (br d, 1H, 3J3′,4′ 8.5 Hz, H-3′), 5.49 (m, 1H, H-2″), 4.6 (d, 2H, 3J1 “,2″≈6.5 Hz, H-1”), 2.61 (s, 3H, H-2), 1.79 (br m, 3H, CH3-prenyl), 1.74 (br s, 3H, CH3-prenyl). 13C NMR (62.9 MHz, CDCl3): δ=200.0 (C═O), 158.3 (C-2′), 138.2 (C-3″), 133.5 (C-4′), 130.3 (C-6′), 128.5 (C-1′), 120.4 (C-5′), 119.1 (C-2″), 112.7 (C-3′), 65.3 (C-1″), 32.0 (C-2), 25.7 (CH3-prenyl), 18.2 (CH3-prenyl). IV (film): γ=3072, 3028 (═CH), 2976 (CH3), 2879 (CH2), 1674 (C═O), 1236 (C—O) cm-1. Elementary quantitative analysis C13H16O2 (204,26): calculated. C 76.44, H 7.89; found C 76.43, H 7.81.

### 10) Obtaining 2-cyan-3-[2-(3-methylbut-2-enyloxy)-phenyl]-but-2-en-nitrile

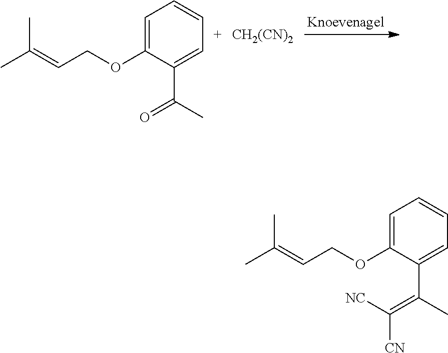

0.01 mol (1.84 g) of 2-(3-methylbut-2-enyloxy)acetophenone, with 9.5 mL of acetic acid, 0.01 mol (0.66 g) of malonitrile, 3.5 g of ammonium acetate and 40 mL of toluene are added. It is placed under reflux with a Dean Stark at a temperature between 120-140° C. for a few hours until it stops forming water. This mixture will be washed with an aqueous solution of NaCl, the organic phase will be dried with anhydrous sodium sulphate, filtered and roto evaporated, where a dark brown oil is formed. This product was purified by a column containing silica gel and a mixture of toluene and ethyl acetate (9:1). C₁₆H₁₆N₂O. MW=252. Yield: 80%. ¹H NMR (250 MHz, DMSO): δ=7.2 (d, 2H, H-3′/H-5′), 6.8 (d, 2H, H-2′/H-4′), 5.2 (m, 1H, H-5), 4.4 (d, 2H, H-4), 2.4 (s, 3H, CH₃(C═C(CN)₂), 1.7 (s, 3H, CH₃), 1.8 (s, 3H, CH₃).

### 11) Obtaining 2-[2-(3-metylbut-2-enyloxy)-phenyl]-4,4-bis-metylsulphanyl-buta-1,3-dien-1,1-dicarbonitrile

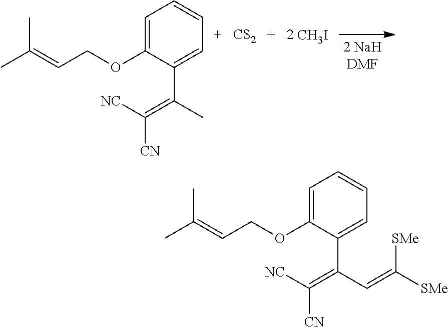

A mixture composed of 1.388 mmol of 2-cyan-3-[2-(3-methylbut-2-enyloxy)-phenyl]-but-2-en-nitrile (0.35 g) and 1.388 mmol of carbon disulfide in 5 mL of absolute dimethylformamide is stirred at room temperature under argon atmosphere. 0.002776 mol of sodium hydride (0.0066 g) are added by stirring under argon atmosphere for eight hours. Subsequently, 2.77 mmol of methyl iodide (173 μL) are added under agitation. After four hours shaking, the reaction mixture is poured into cold water. It is extracted with chloroform and the chloroformic phase is washed three times with water, drying with anhydrous sodium sulphate. Solvent is distilled with the aid of vacuum rotoevaporator and the compound is purified by a silica gel chromatographic column, using as a mobile phase a n-heptane/ethyl acetate mixture ration 1:2 Yield: 80%. ¹H-RMN (CDCl₃, 500 MHz): 1.71 and 1.77 (s, 6H, CH₃ (prenyl), 2.29 and 2.57 (s, 6H, CH₃S), 4.56 (d, 2H, J=6.6 Hz, CH₂), 5.41 (m, 1H, CHCH₂), 6.54 (s, 1H, H2), aromatic protons: 6.94 (dd, 1H, 8.5 Hz; 0.9 Hz, H-3′), 7.00 (m, 1H, 7.5 Hz; 0.9 Hz, H-5′), 7.07 (dd, 1H, 7.5 Hz; 1.9 Hz, H-6′), 7.41 (m, 1H, 8.5 Hz; 7.5 Hz; 1.9 Hz, H-4′) ppm. ¹³C-RMN (CDCl₃, 500 MHz): 165.1 (C-1), 163.4 (C-3), 114.3 (C-2), 78.6 (C-4), 114.4 and 114.2 (CN), 119.6 (CHCH₂), 65.7 (CH₂), 137.8 (HC═C(CH₃)₂, 25.7 and 18.2 (CH₃ prenyl), 17.6 and 16.5 (SCH₃), aromatic carbons: 124.0 (C-1′), 156.0 (C-2′), 112.5 (C-3′), 132.2 (C-4′), 121.1 (C-5′), 129.6 (C-6′) ppm. RMN-DEPT. (CDCl₃): methyl groups (positive signals), 16.490; 17.657 (SCH₃); 18.232; 25,789 (CH₃ prenyl)), (negative signal), 65.725 (CH₂), aromatic carbons: 113.027 (C-3′); 114.249 (C-2); 119.607 (CHCH₂); 121.107 (C-5′); 129.592 (C-6′); 132.279 (C-4′) ppm. IV (capillary registration): 3061.7 and 3027 (═CH and ═CH arom.), 2972 and 2924 (—CH3 Csp³-H), 2876 and 2858 (CH₂), 2219 and 2210 (CN), 1597 and 1579 (C═C) cm⁻¹. C₁₉H₂₀N₂OS₂. MW: 356.10. Quantitative Elementary Analysis: Calculated: 64.04% C; 5.62% H; 7.86% N; 17.97% S. Found: 63.99% C; 5.58% H; 7.84% N; 17.95% S

### 12) Obtaining 4-acetyl Benzoate of Phenyl

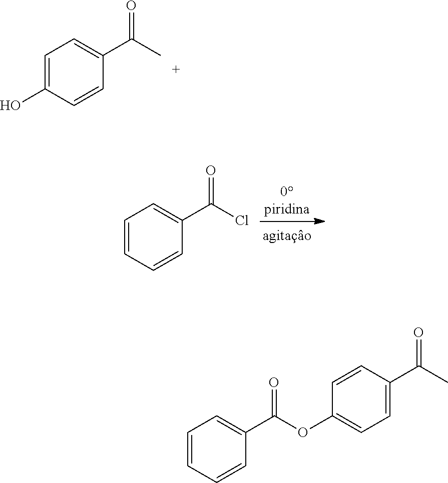

Equimolar quantities of 4-hydroxy-acetophenone and benzoyl chloride in the presence of anhydrous pyridine react at 0° C. under constant agitation to form 4 acetyl benzoate of phenyl. RF of raw material: 0.25 (toluene/ethyl acetate ratio 9/1). RF of the product: 0.5 (toluene/ethyl acetate ratio 9/1). General formula: C₁₅H₁₂O₃. Molecular weight: 240 g/mol. Melting point: 125-130° C. Yield: 2.3 g (97%). Appearance: white solid. IV (KBr): γ=1734 (C═P, ester) 1676 (C═O, Ketone), 1594 (C═C aromatic), 1407 (CH₃ asymmetric deformation), 1452 (CH₃ symmetrical deformation), 1203 (C—O) (substitution in para) cm-1.

### 13) Obtaining 3-Chloro-3-(4-benzoyloxy-phenyl)propenal

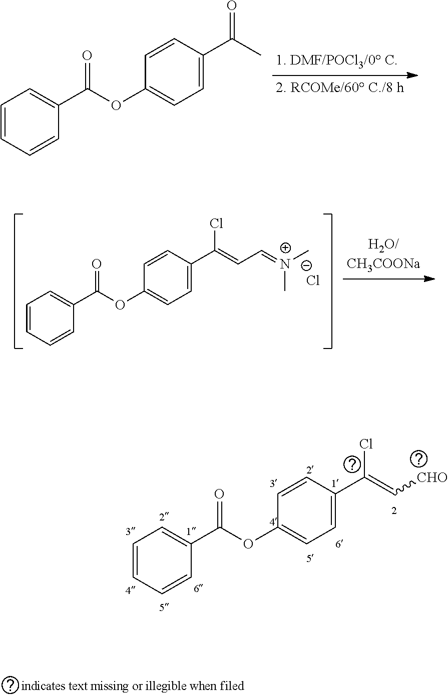

To obtain this product, 0.130 mol of absolute dimethylformamide (9.5 g) (10 mL) is used in a flask and is cooled at a temperature of 0-5° C. 0.0197 mol of phosphorus oxychloride (3.021 g) (2.197 mL) (2197 μL) is added, keeping the temperature in this range under agitation for 10 minutes. Then, 0.01 mol of the 4-acetyl benzoate of phenyl is dissolved in 5 mL of absolute dimethylformamide and this mixture is added, little by little, over the previous one, keeping the temperature in the range 0-5° C. Then it is heated up for 8 hours at 60° C. This mixture is poured over water with ice and sodium acetate. It is extracted with diethyl ether, dried with anhydrous sodium sulphate and the solvent is rotaevaporated. RF of raw material: 0.625 (toluene/ethyl acetate ratio 10/1). RF of the product: 0.5 (toluene/ethyl acetate ratio 10/1). General formula: C₁₆H₁₁O₃Cl. Molecular weight: 286.5 g/mol. Yield: 2 g (70%). Appearance: solid yellow. IV (KBr): γ 2860 and 2656 (CH aldehyde), 1727 (C═O, ester), 1677 (C═O aldehyde), 1502 (C═C, Aromatic), 1210 (C—O), 804, 820 and 836 (probable substitution in para), 707 (intense band of the probable C—Cl binding) cm⁻¹. ¹H NMR (250 MHz, CDCl₃): δ=10.01 (d, 1H, C-1); 6.32 (d, 1H, C-2); 8.4 (d, 2H, H-2′7H-6″); 7.63 (d, 2H, H-3′7H-5″); 7.56 (m, 1H, H-4″); 7.83 (d, 2H, H-27H-6′); 7.34 (d, 2H, H-37H-5′). ¹³C NMR (62.9 MHz, CDCl₃): δ=191.31 (C-1); 171.13 (CO ester); 164.64 (C-3); 153.67 (C-4′); 151.21 (C-1′); 133.97 (C-4″); 133.10 (C-1 ″); 130.25 (C-2″, C-6″); 128.93 (C-3″, C-5″); 128.69 (C-2′, C-6′); 124.42 (C-2); 122.29 (C-3′, C-5′).

DEPT (CDCl₃): δ=191.32 (C-1); 133.99 (C-4″); 130.27 (C-2″, C-6″); 128.70 (C-3″, C-5″); 128.61 (C-2′, C-6′); 124.44 (C-2); 122.31 (C-3′, C-5′).

### 14) Obtaining 4-(1-chloro-4,4-dicyano-buta-1,3-dienyl)phenyl Benzoate

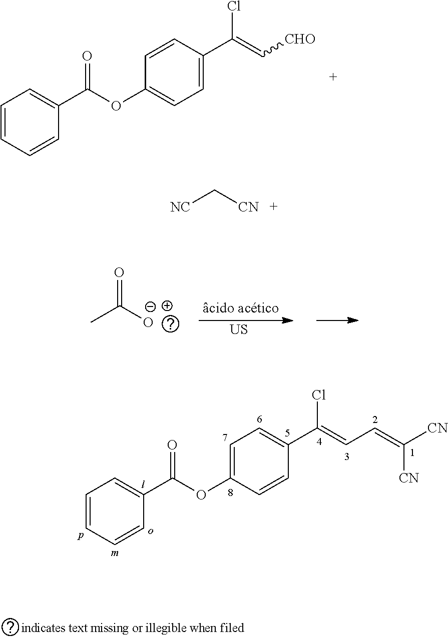

This compound is obtained using the Knoevenagel condensation technique. Starting with 0.004 mol (1.25 g) of 3-Chloro-3-(4-benzyloxy-phenyl)propenal, it is added 5 mL of acetic acid, 0.008 mol (0.6 g) of ammonium acetate and 0.04 mol (2.44 g) of malononitrile This mixture is placed under ultrasonic irradiation for 8 hours at room temperature. After this period, the mixture is poured into water, extracted with ethyl acetate, rinsed with distilled water, dried with anhydrous sodium sulphate and the solvent is roto evaporated. RF of raw material: 0.57 (toluene/ethyl acetate ratio 9/1). RF of the product: 0.42 (toluene/ethyl acetate ratio 9/1). General formula: C₁₉H₁₁N₂O₂Cl. Molecular weight: 334.5 g/mol. Yield: 0.5 g (38%). Appearance: solid yellow. IV (KBr): γ 3116 (═C—H aromatic), 2002 and 2222 (CN), 1741 (C═O, ester), 1201 (C—O), 815, 855 and 872 (probable substitution in para), 699 (intense band of the probable C—Cl binding) cm⁻¹. ¹H NMR (500 MHz, CDCl₃): δ=8.07 (d, 1H, J_{2,3}=11.5, H-2); 7.29 (d, 1H, J_{2,3}=11.5, H-3); 7.88 (m, 1H, H-6); 7.37 (m, 1H, H-7); 8.20 (m, 2H, o-Ph); 7.53 (m, 2H, m-Ph); 7.67 (m, 1H, p-Ph). ¹³C NMR (125.77 MHz, CDCl₃): δ=85.5 (C-1); 154.6 (C-2); 119.4 (C-3); 150.1 (C-4); 132.1 (C-5); 129.2 (C-6); 122.5 (C-7); 154.3 (C-8); 113.2 and 111.5 (CN); 164.5 (COO); 128.8 (i-Ph); 130.3 (o-Ph); 128.7 (m-Ph); 134.1 (p-Ph). DEPT-135,125 MHz, CDCl₃): δ=153.61 (C-2); 133.10 (p-Ph); 129.86 (o-Ph); 129.29 (m-Ph); 128.25 (C-6); 127.74 (C-7); 121.65 (C-3).

### 15) Obtaining 4-(4-acetyl-1-chloro-5-oxo-hexa-1,3-dienyl)phenyl Benzoate

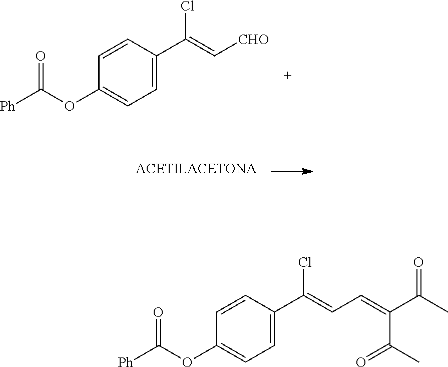 [SMILES: O=C/C=C(\Cl)c1ccc(OC(=O)c2ccccc2)cc1 ; CC(=O)C(=C/C=C(\Cl)c1ccc(OC(=O)c2ccccc2)cc1)C(C)=O]

To obtain this product, 0.01 mol (2.865 g) of 3-chloro-3-(4-benzyloxy-phenyl)propenal with 0.1 mol (10 g) (10.28 mL) of acetylacetone, 0.04 mol (3.08 g) of ammonium acetate and 5 ml of acetic acid were mixed. This mixture is kept under ultrasonic irradiation for 4 hours at room temperature. After this period, the mixture is poured into distilled water and ice. The product precipitates and the precipitate is washed with distilled water. General formula: C₂1H₁₇O₄Cl. Molecular weight: 368.5 g/mol. Yield: 61%. Appearance of product: solid yellow. Thin layer chromatography (CCD): n-hexane 9: Ethyl acetate 1. Rf of 3-chloro-3-(4-benzoyloxy-phenyl)propenal 0.35. Rf of 2-(4-acetyl-1-chloro-5-oxo-hexa-1,3-dienyl)phenyl benzoate 0.75. IV: γ=1734 (C═O ester), 1690 (C═O conjugated ketone), 1592 (C═C aromatic and aliphatic), 1057 (C—O), 704 (C—Cl) cm⁻¹. E. of Weight: (M⁺.+H⁺)=369.08; 391: (M⁺.+Na⁺).

### 16) Obtaining 4-(1-chloro-4-nitro-buta-1,3-dienyl)phenyl Benzoate

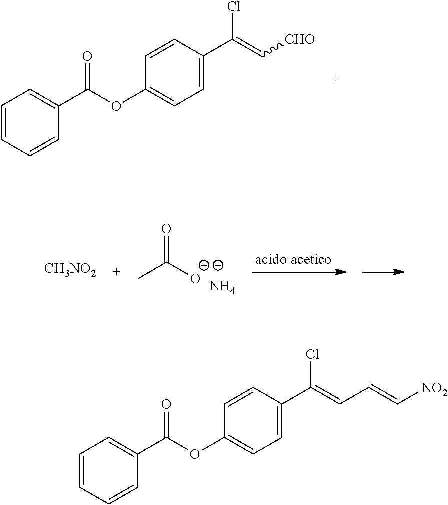

To obtain this product it is mixed 0.004 mol (1.25 g) of 3-Chloro-3-(4-benzyloxy-phenyl)propenal (C5), 5 mL of acetic acid, 0.008 mol (0.6 g) of ammonium acetate and 0.04 mol (2.44 g) (2.15 mL) of nitromethane. This mixture is placed under ultrasonic irradiation for 8 hours at room temperature. After this period, the mixture is poured into distilled water, extracted with ethyl acetate, rinsed with distilled water, dried with anhydrous sodium sulphate and the solvent is roto evaporated. The purification of this compound was performed through a chromatographic column filled with silica gel and using as eluent a mixture formed of toluene/ethyl acetate (9:1). RF of raw material: 0.25 (toluene/ethyl acetate ratio 9/1). RF of the product: 0.75 (toluene/ethyl acetate ratio 9/1). General formula: C₁₇H₁₂NO₄Cl. Molecular weight: 329.5 g/mol. Melting point: 155-157° C. Yield: 0.7 g (54%). Appearance: solid yellow. E.Weight: M⁺. 329 (1.11%), 294 (M⁺.−35; (1.32%), 105 (PhCO, peak base, (100.0%), 77 (M⁺.−252 (36.65%) phenyl.

### 17) Obtaining 3-chloro-3-(4-iodine-phenyl)propenal

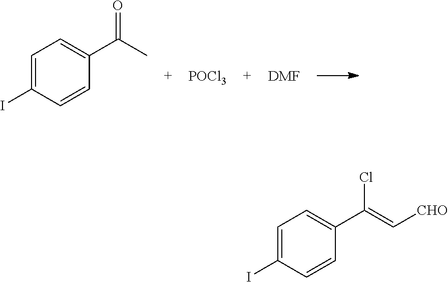

To obtain this product, 0.08 mol (5.84 g) (6.147 mL) of dimethylformamide anhydrous is added drop by drop over 0.044 mol (6.74 g) (4.02 mL) of phosphorus oxychloride, keeping the temperature of 0° C. and constant agitation for 1 hour. After this period, 0.01 mol (2.46 g) of 4-iodoacetophenone diluted in 5 mL of anhydrous dimethylformamide is added, and the temperature is increased to 60° C. and allowed to remain under constant agitation for 6 hours. After 6 hours, the mixture is poured into an ice-cold 10% sodium acetate solution. The product precipitates and the precipitate is washed with distilled water. General formula: C₉H₆ClIO; Molecular weight: 292.5 g/mol; Melting point: 87-93° C.; Yield 85%; Appearance: yellow solid; CCD: toluene 9/ethyl acetate 1; Rf (raw material): 0.77; Rf (Product): 0.85. IV: γ=approximately 2800 small C—H formaldehyde band, 1655 (C═O conjugated aldehyde), 1577 (C═C aromatic and aliphatic.), 1003 (C—I), 708 (C—Cl). E. Weight: M⁺ 292; 257 (M⁺-35) loss of C1; 165 peak base (M⁺- 127) loss of I; 127 (55.98%) I; 77 (5.67%) Ph⁺.

### 18) Obtaining 3-acetyl-6-chloro-6-(4-iodophenyl)-hexa-3,5-diene-2-ona

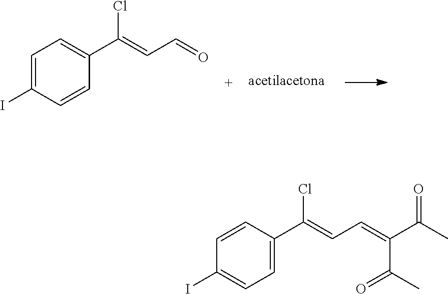 [SMILES: O=C/C=C(\Cl)c1ccc(I)cc1 ; CC(=O)C(=C/C=C(\Cl)c1ccc(I)cc1)C(C)=O]

3-chloro-3-(4-iodinephenyl)acrylate (1.70 mmol, 0.50 g), acetylacetone (17 mmol, 1.75 mL), ammonium acetate (6.80 mmol, 0.52 g) and AcOH (5 mL) were submitted to ultrasonic irradiation for 15 minutes. After this period the mixture is placed in distilled water with ice. The precipitate is filtered and the product is purified by chromatographic column filled with silica gel (petroleum ether/EtOAc 5/1). Formula: C₁₄H₁₂ClIO₂. Weight: 374 g/mol. Yield: 0.553 g (87%). Rf=0.4 (petroleum ether/EtOAc 5/1). PF: 145-150° C. solid yellow. ¹H NMR (250 MHz, DMSO): □□7.85 (dd, 2H, ³J2,6=8.69 Hz, H2/H6), 7.55 (dd, 2H, ³J2,5=8.69 Hz, H2/H5), 7.52 (d, 1H, ³J2′,3′=11.14 Hz, H3′), 7.13 (d, 1H, H2), 2.44 (s, 3H, COCH₃), 2.34 (s, 3H, COCH₃). ¹³C NMR (125 MHz, DMSO): □203.00 (COCH₃), 197.63 (COCH₃), 144.19 (C4′), 144.31 (C1), 137.74 (C2/C6), 135.25 (CCI), 134.55 (C3/C5), 128.72 (C3), 120.93 (C2′), 98.15 (CI), 31.42 (COCH₃), 26.57 (COCH₃). ESI-TOF/MS: [M⁺Na]⁺ calculated for C₁₄H₁₂ClIO2: 374.96433; found: 374.96499. Elementary Analysis: calculated: 90.70% C, 9.30% H; found: 44.522% C, 3.833% H.

### 19) Obtaining 2-cyan-5-chloro-5-(4-iodophenyl)pent-2,4-dienonitrile

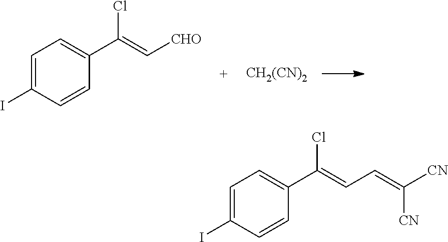

A mixture formed of 3-chloro-3-(4-iodinephenyl)acrylate, (1.70 mmol, 0.50 g), 17 mmol of malonitrile (1.12 g), 6.08 mmol of ammonium acetate (0.524 g), and 5 mL of acetic acid was irradiated by ultrasound for 2.5 hours at room temperature. The mixture is poured into water and the product precipitates. The product was purified by a chromatographic column using as eluents a mixture of toluene and ethyl acetate (10/1).

Chart 1: Mass spectrum of the compound in CG-Masses

### 20) Obtaining 4-chloro-4-(4-iodinepheny)-1-nitro-buta-1,3-diene

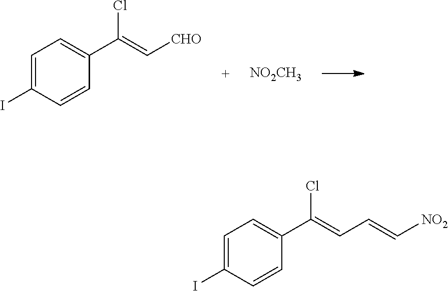

A mixture formed of 3-chloro-3-(4-iodinephenyl)acrylate, (1.70 mmol, 0.50 g), 17 mmol of nitromethane (5.35 mL), 6.08 mmol of ammonium acetate (0.524 g), and 5 mL of acetic acid was submitted to irradiation by ultrasound for 2.0 hours at room temperature. The mixture is poured into water and the product is purified by chromatographic column filled with silica gel and using as mixture eluent hexane/ethyl acetate 15-1. A yellow solid was isolated.

Chart 2: Mass spectrum of the compound in CG-Masses

### 21) Obtaining 5-chloro-2-(3,4-dimethoxy-benzoyl)-5-(4-iodine-phenyl)-penta-2,4-dienonitrile

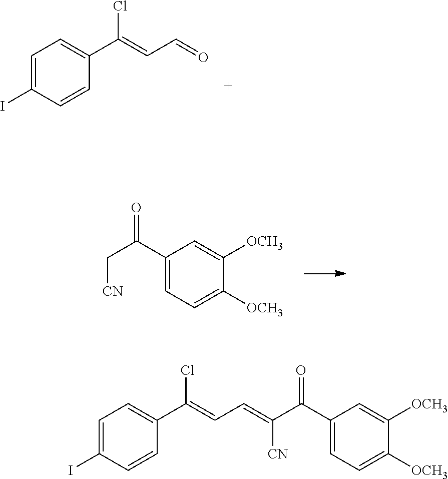 [SMILES: O=C/C=C(\Cl)c1ccc(I)cc1 ; COc1ccc(C(=O)CC#N)cc1OC ; COc1ccc(C(=O)/C(C#N)=C/C=C(\Cl)c2ccc(I)cc2)cc1OC]

3-Chloro-3-(4-iodinephenyl)acrylate, (0.58 mmol, 0.17 g), 3,4-dimethoxybenzylacetonitrile (1.16 mmol, 0.24 mL), ammonium acetate (2.32 mmol, 0.14 g) and AcOH (5 mL) were submitted to ultrasonic irradiation for 1 hour. After this period the mixture is placed in distilled water with ice, EtOAc is extracted and dried. The organic phase is concentrated in vacuum. The crude product is filtered using chromatographic column filled with silica gel (petroleum ether/EtOAc 5/1). Formula: C₂₀H₁₅ClINO₃. Weight: 479 g/mol. Yield: 0.135 g (69%). Rf=0.36 (petroleum ether/EtOAc 5/1). PF: 70-75° C. Solid yellow. ¹H NMR (300 MHz, DMSO): □8.05 (d, 1H, ³JH2′/H3′=11.33 Hz, H3′), 7.93 (d, 2H, ³JH3″,H5″/H2″,H6″=8.69 Hz, H3″/H5″), 7.67 (d, 2H, H2″/H6″), 7.55 (dd, 1H, ³JH2′″/H6′″=2.08 Hz, H6′), 7.45 (d, 1H, ³JH5′/H2′″=8.50 Hz, H2′), 7.41 (d, 1H, H2′″), 7.16 (d, 1H, H5′″), 3.88 (s, 3H, OCH₃), 3.83 (s, 3H, OCH₃). ¹³C NMR (300 MHz, DMSO): □ 186.60 (CO), 153.57 (C3′″), 148.66 (C4′″), 148.53 (C3′), 146.35 (C1′), 138.01 (C3″/C5″), 134.56 (C1″), 129.04 (C2″/C6″), 127.76 (C1″), 124.13 (C6′), 120.99 (C2′), 115.57 (C═N), 114.49 (C4″), 111.75 (C2′″), 110.97 (C5′″), 99.72 (C4′), 55.86 (OCH₃), 55.62 (OCH₃). ESI-TOF/MS: [M⁺Na]⁺ calculated for C₂₀H₁₅ClINO₃: 479.98579; found: 479.98532.

### 22) Obtaining (E)-4-(3,4-dimethoxyphenyl)but-3-en-2-ona

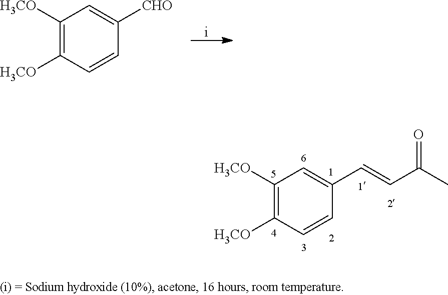

Acetone (45 mmol, 3.3 mL) and 10% and NaOH aqueous solution (2 mL) were added to a three-neck flask coupled to a separatory funnel. Then drop by drop it was added, with the aid of the separatory funnel, solution of 3,4-dimethoxybenzaldehyde, (9 mmol, 1.5 g) in ethanol. The mixture was shaken for 16 hours at room temperature. After this period, the mixture was poured into water and ice, extracted with EtOAc, washed with water, dried and concentrated with vacuum (ADAEVA, 2000). The product purification was performed by chromatographic column filled with silica gel (petroleum ether/EtOAc, 2/1). Formula: C12H1403. Weight: 206 g/mol. Yield: 0.80 g (43%). Rf=0.72 (petroleum ether/EtOAc/EtOAc, 2/1). Solid yellow. ¹H RMN (300 MHz, DMSO): □ 6.70 (d, 1H, ³J_{1′,2′} =16.24 Hz, H1′), 7.33 (d, 1H, ³J_{2,3}=1.89 Hz, H3), 7.25 (dd, 1H, ³J_{2,6}=8.31 Hz, H2), 7.00 (d, 1H, H6), 6.74 (d, 1H, H2′), 3.80 (d, 6H, 2×OCH₃), 2.30 (s, 3H, COCHs). ¹³C RMN (300 MHz, DMSO): □ 197.86 (C═O), 151.00 (C4), 148.97 (C5), 143.51 (C1′), 127.13 (C1), 125.18 (C2′), 123.03 (C2), 111.58 (C6), 110.36 (C3), 55.55 (OCH₃), 27.17 (COCHs). ESI-TOF/MS: [M⁺Na]⁺ calculated for C₁₂H₁₄O₃: 229.08352; found: 229.08376.

### 23) Obtaining (4E)-3-chloro-5-(3,4-dimethoxyphenyl)penta-2,4-dienal

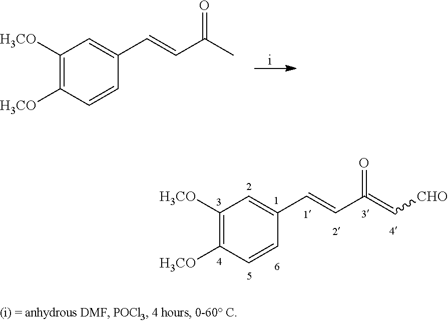

In a three-neck flask coupled to a separatory funnel and reflux condenser, anhydrous DMF (12.26 mmol, 0.94 mL) was added drop by drop over phosphorus oxychloride (6.73 mmol, 0.63 mL) at 0° C. temperature. After full separation the mixture was stirred 1 hour at room temperature. At this point the reaction was cooled again to 0° C. with the aid of ice bath, and then a solution of (E)-4-(3,4-dimethoxyphenyl)but-3-en-2-ona (1.52 mmol, 0.31 g) in 5 mL of anhydrous DMF was added drop by drop with the aid of the separatory funnel. The mixture was shaken for 4 hours at temperature of 60° C. After this period, the reaction was cooled slowly at room temperature and poured into concentrated sodium acetate solution, extracted with EtOAc, dried and vacuum concentrated (LIEBCHER, 1976). The final product was purified by silica gel chromatographic column (petroleum ether/EtOAc, 2/1). Formula: C₁₃H₁₃ClO₃. Weight: 252 g/mol. Yield: 0.216 g (56%). Rf=0.72 (petroleum ether/EtOAc 2/1). Solid yellow. ¹H RMN (300 MHz, DMSO): □ 10.32 (dd, 1H, ³J_{CHO,4′}=7.55 Hz, CHO_{E}), 10.20 (dd, 1H, ³J_{CHO,4′}=7.36 Hz, CHO_{Z}), 7.48-7.38 (m, 2H, ³J_{2′,4′}=7.37 Hz, ³J_{2′, 2}=10.39 Hz, H1′/H2′), 7.35 (d, 1H, ³J_{2,3}=1.89 Hz, H5), 7.26 (dd, 1H, ³J_{2,6}=8.31 Hz, H6), 7.02 (d, 1H, H2), 6.45 (d, 1H, H4′_{Z}), 6.22 (dd, H4′_{E}), 3.81 (d, 6H, OCH₃). ¹³C RMN (300 MHz, DMSO): □ 190.93 (CHO), 150.84 (C4), 149.37 (C5), 149.02 (C3), 139.37 (C1′), 127.71 (C1), 125.42 (C4′), 123.22 (C2′), 122.72 (C2) 111.72 (C6), 110.47 (C3), 55.59 (OCH₃). Elementary Analysis: calculated: 61.79% C, 5.19% H; found: 60.584% C, 5.227% H.

### 24) Obtaining 3-((2Z,4E)-3-chloro-5-(3,4-dimethoxyphenyl)penta-2,4-dien-ylidene)pentane-2,4-dione

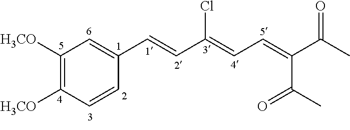 [SMILES: COc1ccc(/C=C/C(Cl)=C/C=C(C(C)=O)C(C)=O)cc1OC]

(4E)-3-chloro-5-(3,4-iodinephenyl)penta-2,4-dienal (0.65 mmol, 0.16 g), acetylacetone (6.55 mmol, 0.67 mL), ammonium acetate (2.62 mmol, 0.20 g) and AcOH (5 mL) were submitted to ultrasonic irradiation for 1 hour. After this period the mixture was placed in distilled water with ice. The product is filtered and the product is purified by chromatographic column filled with silica gel (petroleum ether/EtOAc 1/1). Formula: C₁₈H₁₉ClO₄. Weight: 334 g/mol. Yield: 0.135 g (62%). Rf=0.22 (petroleum ether/EtOAc 1/1). PF: 84-87° C. solid yellow. ¹H NMR (250 MHz, DMSO): □ 7.53 (d, 1H, ³J4′,5′=11.71 Hz, H4), 7.40 (d, 1H, ³J1′,2′=15.30 Hz, H1′), 7.32 (d, 1H, H3), 7.20-7.14 (m, 2H, ³J2,3=1.70, ³J2,6=8.31 Hz, Hz, H2/H2′), 6.97 (d, 1H, H6), 6.83 (d, 1H, H5), 3.80 (d, 6H, 2×OCH₃), 2.41 (s, 3H, COCH₃), 2.32 (s, 3H, COCH₃). ¹³C NMR (300 MHz, DMSO): □ 203.20 (COCH₃), 197.25 (COCH₃), 150.31 (C4), 149.04 (C5), 142.52 (C(COCH₃)₂), 142.42 (C3), 136.66 (C2), 134.80 (C4′), 128.31 (C1), 124.32 (C1′), 122.58 (C5′), 122.35 (C2), 111.67 (C6), 109.75 (C3), 55.54 (2×OCH₃), 31.44 (COCH₃), 26.37 (COCH₃). ESI-TOF/MS: [M⁺Na]⁺ calculated for C₁₈H₁₉ClO₄: 357.08641; found: 357.08668. Elementary Analysis: calculated: 64.57% C, 5.72% H; found: 64.073% C, 5.868% H.

### 25) Obtaining 3-chloro-3-(3,4-dimethoxyphenyl)-propenal

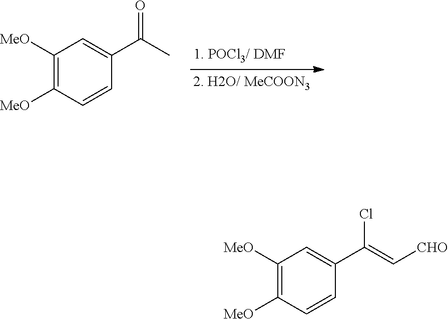

In a three-neck flask of 250 mL, with a reflux condenser with calcium chloride tube, dropping funnel and wash with ice and salt, 0.004 mol (0.6132 g; 0.366 mL) are placed of phosphorus oxychloride, Argon flow is passed and agitated for 15 minutes, next, dropping 0.004 mol (0.2924 g; 0.308 mL) of absolute DMF, keeping the Argon flow, with agitation for 1 hour. Then, 0.001 mol (0.180 g) of 3,4-dimethoxy-acetophenone are dissolved in 5 mL of absolute DMF and slowly dripped. Shaking is kept with cooling (ice bath with salt) for 1 hour. Chopped ice and saturated sodium acetate solution are added, maintaining agitation for 15 minutes. The solid that precipitates is filtered and washed with water twice. The product is purified by a silica chromatographic column saturated with a mixture of toluene:ethyl acetate (7/3). Yield 56%; Appearance: Yellow powder; CCD: Toluene: ethyl acetate 70/ethyl acetate 30; Rf (product): 0.538.

Mass spectrum: M+. 226; 225 (M+−1); 211 (M+−15) (CH₃); 195 (peak base) (M+−31) (OMe); 183 (211-28=CO); 163+C6H2-CCI═CH—CHO (195-32=MeOH). RMN-1H spectrum (300 MHz-DMSO-d6): δ=10.11 (d, 1H, CHO); 9.42 (d, 1H, CHO), (these aldehyde group signals are ratio 1:0:0.071:0:0.07); 7.02 (d, 1H, H2); 7.54 (dd, 1H, H6′); 7.42 (d, 1H, H2′); 7.09 (d, 1H, H5′); 3.841 (s, 3H, MeO); 3.839 (s, 3H, MeO); 3.833 (s, 3H, MeO); 3.810 (s, 3H, MeO) ppm.

### 26) Obtaining 2,4 bis(benzoyloxy)acetophenone

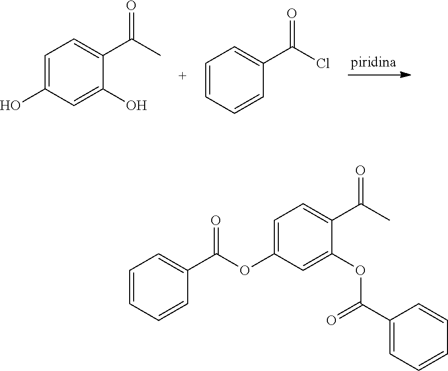

In a flask, 0.01 mol of 2,4-dihydroxyacetophenone (1.52 g) was placed with 10 mL of anhydrous pyridine at 0° C. under constant agitation. After that, 0.02 mol (2.81 g, 2.32 mL) of benzoyl chloride was dripped. The mixture was kept at 0° C. under constant agitation for 3 hours. After this period, the mixture was filtered and rinsed with plenty of distilled water. General formula: C₂₂H₁₆O₅; Molecular weight: 360 g/mol; Melting point: 74-74° C.; Yield 66%; Appearance: yellow solid; CCD: toluene 9/ethyl acetate 1; Rf (raw material): 0.375; Rf (Product): 0.75. IV (KBr): γ 3114 (═C—H), 1744 (C═O, ester), 1670 (C═O, Ketone conjugated with the aromatic ring), 1451 (CH₃ asymmetric deformation), 1314 (CH₃ symmetrical deformation), 1241 (C—O), 824 (substitution in para), 705 (intense replacement ortho band) cm⁻¹.

Obtaining chlorovinyl aldehyde from benzoylated hydroxy acetophenones with DMF/POCl₃ through the reaction of Vilsmeier-Haack

### General Technique:

To obtain these product, it is started from 0.130 mol of absolute dimethylformamide (9.5 g) (10 mL) in a flask and is cooled at a temperature of 0-5° C. 0.0197 mol of phosphorus oxychloride (3.021 g) (2.197 mL) (2197 μL) is added, keeping the temperature in this range under agitation for 10 minutes. Then, 0.01 mol of the corresponding benzoylated hydroxy acetophenone is dissolved in 5 mL of absolute dimethylformamide and this mixture is added, little by little, over the previous one, keeping the temperature in the range 0-5° C. Then it is heated up for 8 hours at 60° C. This mixture is poured over water with ice and sodium acetate. It is extracted with diethyl ether, dried with anhydrous sodium sulphate and the solvent is rotaevaporated.

### 27) Obtaining 3-Chloro-3-(2,4-benzoyloxy-phenyl)propenal

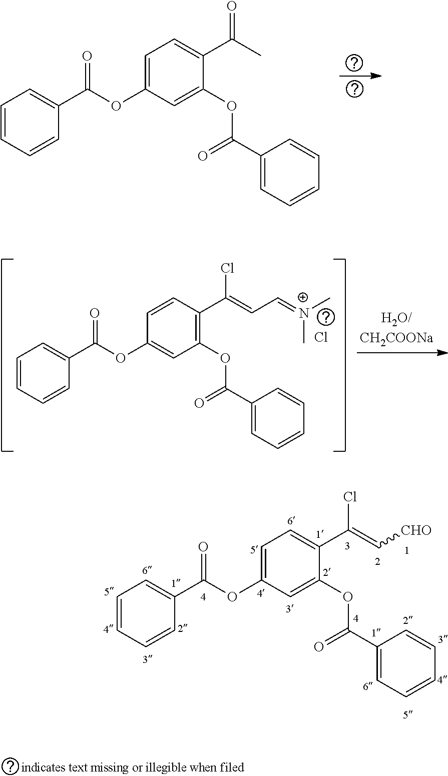

RF of raw material: 0,625 (toluene/ethyl acetate ratio 9/1). RF of the product: 0.75 (toluene/ethyl acetate ratio 9/1). General formula: C₂₃H₁₅O₅Cl. Molecular weight: 406.5 g/mol. Yield: 2.7 g (67%). Appearance: solid light yellow. ¹H NMR (250 MHz, CDCl₃): δ=10.02 (d, 1H, H-1); 9.40 (d, 1H, H-1); set of multiplets between 7.21 and 8.17 aromatic protons, being the multiplet in 8.10 (H-2″/H-6″); multiplets between 7.21 and 7.60 (H-3″/5″, H-4″ H-6′/H-5′/H-3′), 6.42 (d, 2H, C-2). ¹³C NMR (62.9 MHz, CDCl₃): δ=189.687 (C-1); 188.104 (C-1); 163.358 (C-4); 163.296 (C-4); 152.262 (C-3); 152.131 (C-3); 147.522 (C-4′); 146.336 (C-2′); 133.223 (C-4″); 133.223 (C-2); 133.058 (C-1″); 131.124 (C-2″/C-6″); 128.101 (C-3″/C-5″); 127.833 (C-6′); 127.781 (C-1′); 118.834 (C-5′); 116.628 (C-3′).

DEPT (CDCl₃): δ=189.669 (C-1); 188.018 (C-1); 133.225 (C-4″); 133.060 (C-2); 131.117 (C-2″/C-6″); 129.293 (C-3″/C-5″); 128.101 (C-6′); 118.836 (C-5′); 116.627 (C-3′).

IV (KBr): γ=3077 (═C—H), 2756 and 2890 (aldehyde CH), 1745 (C═O, ester), 1672 (aldehyde C═O), 814 (probable substitution in para), 704 (probable replacement in ortho) cm⁻¹.

### 28) Obtaining (E)-4-(4-hydroxy-3-methoxyphenyl)but-3-en-2-ona

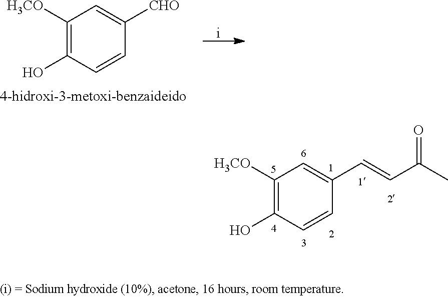

Acetone (50 mL) and 10% and NaOH aqueous solution (2 mL) were added to a three-neck flask coupled to a separatory funnel. Then drop by drop it was added, with the aid of the separatory funnel, solution of 4-hydrozymethoxybenzaldehyde, (10 mmol, 1.52 g) in ethanol. After 72 hours of constant agitation at room temperature, the mixture was poured into water and ice, extracted with EtOAc, washed with water, dried and concentrated with vacuum (ADAEVA, 2000). The product purification was performed by chromatographic column filled with silica gel (petroleum ether/EtOAc, 2/1). Formula: C₁₁H₁₂O₃. Weight: 192 g/mol. Yield: 1.91 g (100%). Rf=0.36 (petroleum ether/EtOAc 2/1). P.F.: 120-124° C. Solid yellow. ¹H RMN (500 MHz, DMSO): □ 9.62 (s, 1H, OH), 7.51 (d, 1H, ³J_{H1′/H2′}=16.24 Hz, H1′), 7.30 (d, 1H, H2), 7.13 (dd, 1H, ³J_{H5/H6}=8.12 Hz, H6), 6.81 (d, 1H, H5), 6.67 (d, 1H, H2′), 3.82 (s, 3H, OCH₃), 2.29 (s, 3H, COCH₃). ¹³C RMN (300 MHz, DMSO): □ 197.78 (CO), 149.38 (C4), 147.94 (C3), 143.90 (C1′), 125.84 (C1), 124.31 (C2′), 123.22 (C6), 115.61 (C5), 111.26 (C2), 55.62 (OCH₃), 27.13 (COCH₃). ESI-TOF/MS: [M⁺Na]⁺ calculated for C₁₁H₁₂O₃: 215.06787; found: 215.06780.

### 29) Obtaining (1E,4E,6Z,8E)-7-chloro-1-(4-hydroxy-3-methoxyphenyl)-9-(3,4-dimethoxyphenyl)nona-1,4,6,8-tetraen-3-ona

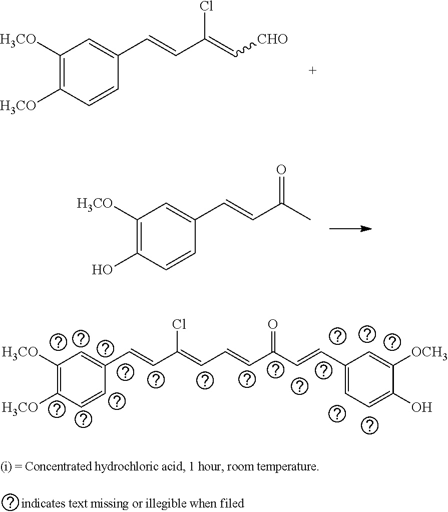 [SMILES: COc1ccc(/C=C/C(Cl)=CC=O)cc1OC ; COc1cc(/C=C/C(C)=O)ccc1O ; COc1cc(/C=C/C(=O)/C=C/C=C(Cl)/C=C/c2ccc(OC)c(OC)c2)ccc1O]

In a round-bottomed flask, the compounds (4E)-3-chloro-5-(3,4-dimethoxyphenyl)penta-2,4-dienal (0.4 mmol, 0.1 g), (E)-4-(4-hydroxy-3-methoxyphenyl)but-3-en-2-ona (0.36 mmol, 0.07 g) and concentrated HCl (3 mL) were added and irradiated for 1 hour at room temperature. After this period, the mixture was poured into water and ice, extracted with EtOAc, dried and vacuum concentrated (ADAEVA, 2000). The product was purified by chromatographic column filled with silica gel (petroleum ether/EtOAc, 2/1). Formula: C₂₄H₂₃ClO₅. Weight: 426 g/mol. Yield: 0.07 g (46%). Rf=0.33 (petroleum ether/EtOAc 2/1). P.F.: 120-124° C. Yellow crystal (DMSO/H20). ¹H RMN (250 MHz, DMSO): □ 7.70-7.58 (m, 2H), 7.36 (d, 1H), 7.30-7.24 (m, 2H), 7.21-7.14 (m, 2H), 7.10 (d, 1H), 7.04 (d, 1H), 6.94 (t, 2H), 6.86 (d, 1H), 6.82 (d, 1H), 3.82 (s, 3H, OCH₃), 3.80 (s, 3H, OCH₃), 3.77 (s, 3H, OCH₃). ¹³C RMN (300 MHz, DMSO): □ 187.64 (CO), 149.92, 149.67, 148.95, 147.92, 143.64, 139.50, 135.02, 134.81, 130.87, 128.46, 126.61, 126.08, 124.28, 123.65, 123.19, 121.50, 115.62, 111.70, 111.50, 109.95, 59.68, 55.66, 55.49. ESI-TOF/MS: [M⁺Na]⁺ calculated for C₂₄H₂₃ClO₅: 427.13068; found: 427.13064.

### Cytotoxic Studies Performed

### Tumor Cell Line Culture

The tumor line of murine melanoma B16F10 was donated by the Ludwig-Switzerland Institute. Adherent cell suspensions of B16F10 cells were obtained for all experimental procedures by treating culture flasks with trypsin 0.2% for 5 minutes and inactivated with 10% fetal bovine serum. The detached cells were centrifuged twice, resuspended in supplemented RPMI-1640 medium. The count was performed in Malassez chamber, and the cell concentration was adjusted in 5×105 cells/ml, in RPMI-1640 culture medium supplemented with 10% fetal bovine serum and 7 ug polymyxin-B (Sigma Chemical Company, St Louis Mo.-USA).

Cell viability was determined by the Trypan Blue exclusion test, being greater than 95% of viable cells. The cells were grown in plates with 96 well plate flat bottoms (Corning), at a concentration of 2×105 cells, maintained for 24 hours in a CO₂ oven at 37° C. After this period, the plates were centrifuged for 5 minutes, at 2000 rpm at 4° C., the supernatant discarded and added different concentrations of the compounds, diluted in RPMI-1640 culture medium, supplemented and increased by 7 μg of Polymyxin-B (Sigma Chemical Company). After treatment with the various diluted compounds in complete culture medium, the cells were incubated with 0.5 mg/mL of MTT reactive.

### Culture and Obtaining Normal Human Skin Fibroblasts.

Fragments of cutaneous biopsies from patients treated by the Dermatology Service of the Clinic Hospital of the Medicine School of USP were removed for routine diagnostic procedures and part of the surgical flap edge immersed in Eagle culture medium modified by Dulbecco's (DMEM, Sigma Chemical Co., St. Louis, Mo., USA) added of 10% fetal bovine serum (Cultilab Ltda., Campinas-SP), 125 mg/ml ampicillin G and 50 ng/ml amphotericin B at 4 C and kept at 4 C for a maximum of 16 hours.

The fragments were washed three times in the same solution, cut into fragments of approximately 1 mm³ and transferred to 25 cm² culture flasks, kept at 37 C, humid atmosphere containing 5% CO₂. Cell growth monitored daily, photo documented in inversion microscopy and culture medium exchanged every 2 or 3 days, according to cell metabolism.

After 7 days, the first change in the culture medium of the flasks was performed once cell growth started, the culture medium was renewed every three days. Plastic flasks were used for cultivation of 25 cm² of cultivated area, where more 3 ml of DME medium containing 10% fetal bovine serum and 1% of antibiotic-antimycotic solution will be added.

### Colorimetric Assay for the Determination of Cellular Viability (MTT)

The cell viability test was performed to verify the effect of the various compounds on the various concentrations previously established: on the lines of tumor cells B16F10 and normal human fibroblasts. The MTT method consists of a cell viability test that measures the activity of mitochondrial dehydrogenase. MTT is a colorimetric method based on the capacity of living cells to reduce salt 3-(4,5-di-methylazole-2-IL)-2,5-diphenyl tetrazolium bromide in the formazan product (Mosmann, 1983). After the cells plating (concentration of 1×104 per well of the culture plate) in RPMI-1640 medium and 10% SFB medium, they were maintained at 37° C. for periods of 24, 48, 72 and 96 hours. Four hours before the set time was completed, MTT 20 μL (Sigma) was added (final concentration of 10 μg/mL). The plates were kept in the oven for the remaining four hours. After the stipulated time, 180 μL of the supernatant were removed from each well and then added 150 μL of dimethyl sulfoxide (Sigma) homogenizing well for the complete dissolution of the salt crystals formed by the mitochondrial metabolism resulting in a staining.

The 96-well plate was read by the spectrophotometer (Spectra MAX-190) using the wavelength of 570 nm. The results were analyzed by absorbance of each well. The percentage of viability was obtained using the following formula: [(Absorbance of treated cells/Absorbance of untreated cells)×100]. The experiments were carried out in quadruplicate.

### Dilution of the Samples.

The samples of the compounds obtained by synthesis chemistry were diluted in DMSO solution at the final concentration of 10%.

### Cytotoxicity Results of Synthesized Compounds:

In attempts to find new O-prenylated compound with better antitumor properties (see Quincoces, J. et al Europ. J. Med. Chem. 41 (2006) 401-407), the following 4p products have been synthesized: 4-(3-Methylbut-2-enyloxy)acetophenone, 5p: 2-Cyano-3-methyl-3-[4-(3-methylbut-2-enoyloxy)-phenyl]acrylonitrile and 6p: 4,4-Bis-ethylsulfanyl-2-[4-3-methylbut-2-enyloxy)-phenyl]1,1-dicarbonitrile:

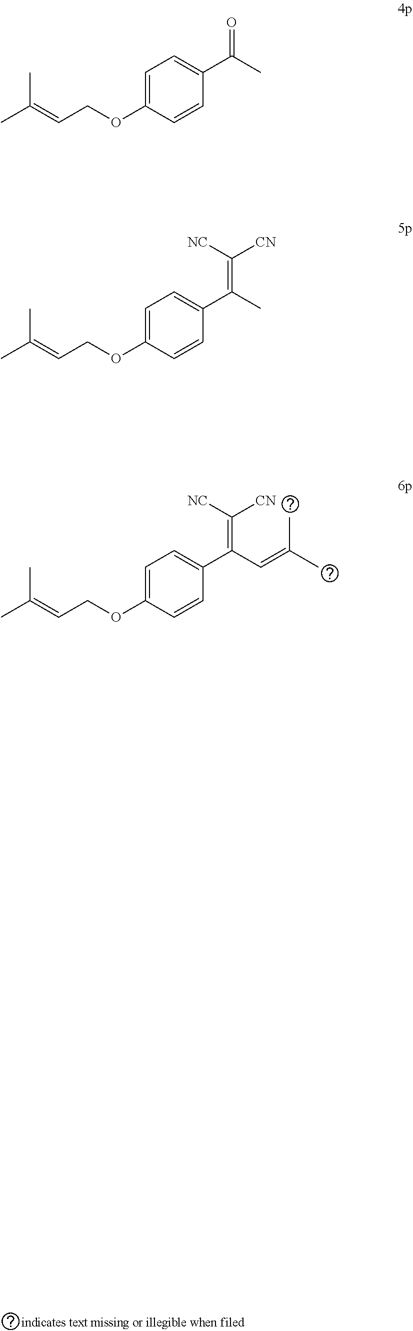

TABLE 1
In vitro cytotoxicity of O-prenylated compounds
Table 1: In vitro cytotoxicity of O-prenylated compounds

| | | IC50 μM | | | | | |
|---|---|---|---|---|---|---|---|
| | | Compounds | | | | | |
| Cell line | Origin | 4 p | | 5 p | | 6 p | |
| CaCo2 | Colon | 759 | μM | 163 | μM | 8.07 | μM |
| HT-29 | Colon | 455 | μM | 92 | μM | 3.9 | μM |
| Colo205 | Colon | 1030 | μM | 149 | μM | 14.3 | μM |
| SW620 | Colon | 980 | μM | 134 | μM | 11.7 | μM |
| CRO2B | Carcinoid | 666 | μM | 149 | μM | 6.51 | μM |
| MIAP2 | Pancreas | 823 | μM | 155 | μM | 3.9 | μM |
| BxPC3 | Pancreas | 725 | μM | 160 | μM | 24.2 | μM |
| PANC1 | Pancreas | 759 | μM | 177 | μM | 50 | μM |

The synthesized O-prenylated compounds were tested in vitro to determine their possible cytotoxic activity. The 4p compound: 4-(3-methyl-but-2-enyloxy)acetophenone, showed no cytotoxic activity, however the 5p compound: 2-cyan-[4-(3-methylbut-2-enyloxy)-phenyl]-3-methyl-acrylonitrile, obtained by Knoevenagel condensation from compound 4, exhibited a better cytotoxic activity against the eight lines of carcinogenic cells tested. The reaction of compound 5 with carbon disulfide and ethyl iodide in basic medium allowed the obtaining of a 6P-composed push-pull butadiene: 4,4-bis-ethylsulfanyl-2-[4-(3-methylbut-2-enyloxy)-phenyl]-1,1-dicarbonitrile, with higher electronic conjugation, which had a potent cytotoxic activity against all the lines of human carcinoma tested. The CI50 values of this compound were equal to or lower than 50 μm, being mainly effective against the four lines of colon tested and standing out against the lines of pancreas studied. The low cytotoxic activity of the 4p compound was not expected, since its isomer, 2-(3-methylbut-2-enyloxy)acetophenone, had already shown a good cytotoxic activity against NC1460 lung carcinoma cells, UACC62 melanoma, MCF breast and multiple NCIADR drugs resistant ovary.

Cytotoxicity of the 2-(3-methylbut-2-enyloxy)acetophenone prenylated compound against human carcinogen lines NC1460 (lung), UACC62 (melanoma), MCF7 (normal Breast), NCIADR (multiple drug resistant Ovary).

| | Tested human cell lines CI50 (μM) | | | |
|---|---|---|---|---|
| Compound | NCI460 | UACC62 | MCF7 | NCIADR |
| 2-(3-methylbut-2- | 122 | 220 | 122 | 39.2 |
| enyloxy)acetophenone | | | | |

This result indicates that the spatial structure of the substituents is another factor to be considered in the choice of compounds to be synthesized.

Also, here in this other example, we can observe that as the electronic conjugation C═C increases, better cytotoxic results are obtained.

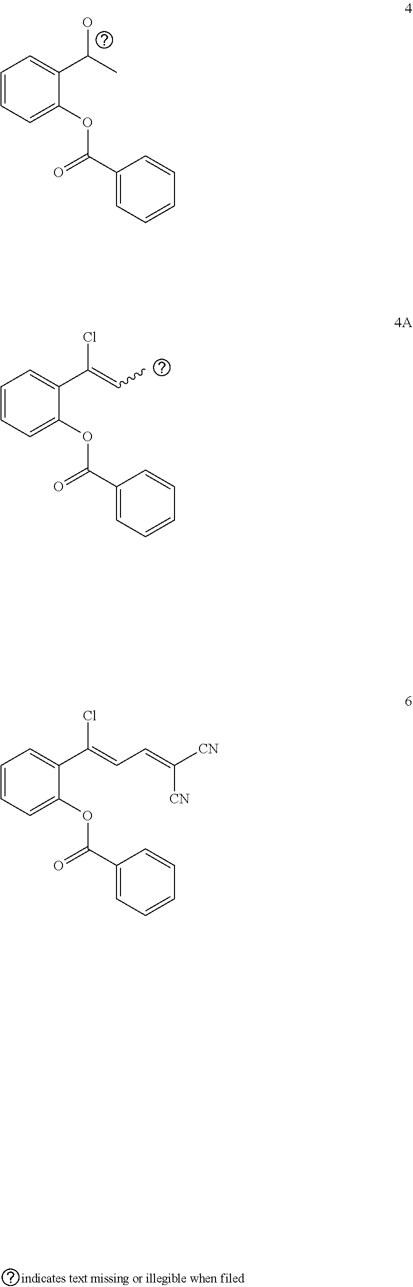

TABLE 2
In vitro cytotoxic activity shown by 2-(1-
chloro-4,4-dicyano-1,3-dienyl)phenyl benzoate.

| Cell line | Origin | IC50 μM |
|---|---|---|
| CaCo2 | Colon | 9.25 |
| HT-29 | Colon | 4.47 |
| Colo205 | Colon | 16.4 |
| SW620 | Colon | 13.4 |
| CRO2B | Carcinoid | 7.46 |
| MIAP2 | Pancreas | 4.47 |
| BxPC3 | Pancreas | 27.7 |
| PANC1 | Pancreas | 57.3 |

The synthesis of push-pull butadienes can also be carried out from the compound 4: 2-acetyl benzoate of phenyl Underthe reaction conditions of Vilsmeier-Haack, compound 4 reacts with POCl₃ and DMF, forming with good yield, the compound 4A: benzoate of 2-(1-chloro-3-oxo-propenyl)-phenyl. The reaction of this P-aryl-p-chloro-acrolein with malonitrile under the Knoevenagel-Cope reaction conditions allows the obtaining of good yields of the butadiene push-pull compound 6: 2-(1-chloro-4,4-dicyano-buta-1,3-dienyl)phenyl benzoate In cytotoxic studies performed with this compound 6 we found a potent in vitro activity against all tumor cell lines of human carcinoma tested (See Table 2). Compound 4 was inactive before these tumor lines, however compound 4A had a cytotoxic activity seven times lower than that shown by compound 6. Here, also, it is confirmed that with an increase in the C═C electronic conjugation, cytotoxic activity is increased. All these cytotoxic results obtained with push-pull butadienes: 4,4-bis-ethylsulphanil-2-[4-(3-methylbut-2-enyloxy)-phenyl]-buta-1,3-diene-1,1-dicarbonitrile; 2-(1-chloro-4,4-dicyano-buta-1,3-dienyl)phenyl benzoate and derivatives encouraged us to extend the family of these push-pull butadienes, with a new purpose of generating a patent application.

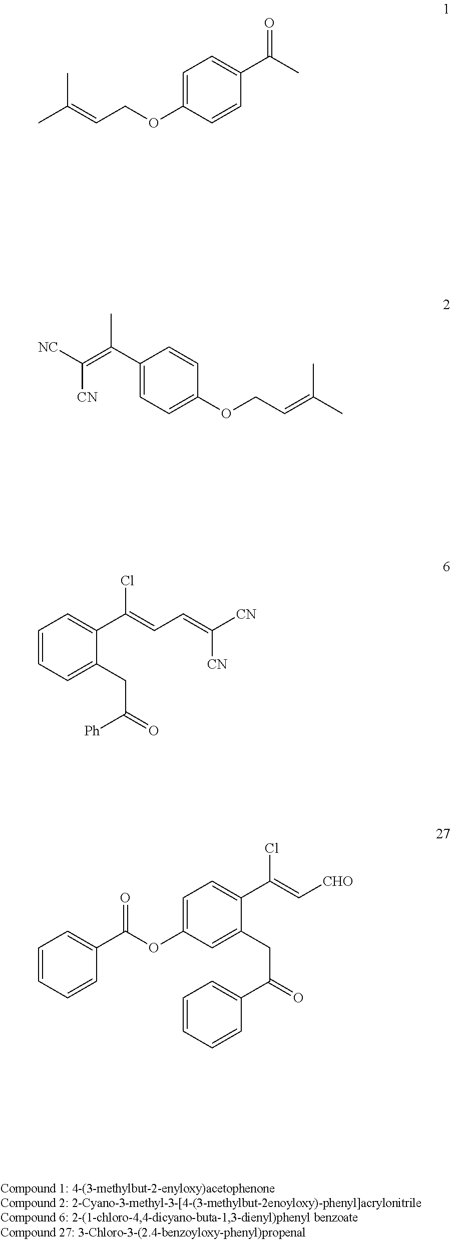 [SMILES: CC(=O)c1ccc(OCC=C(C)C)cc1 ; CC(C)=CCOc1ccc(C(C)=C(C#N)C#N)cc1 ; N#CC(C#N)=C/C=C(\Cl)c1ccccc1CC(=O)c1ccccc1 ; O=C/C=C(\Cl)c1ccc(OC(=O)c2ccccc2)cc1CC(=O)c1ccccc1]

TABLE 3
Antitumor activity of prenylated and benzoyl compounds

CI50% in □M

| Line | 4 | 5 | 46 | 9 |
|---|---|---|---|---|
| Caco2 | 759 | 163 | 61.6 | 9.25 |
| HT29 | 455 | 92 | 56.6 | 4.47 |
| MIAP2 | 823 | 156 | 123 | 4.47 |
| BxPC3 | 725 | 160 | 123 | 27.7 |
| Colo205 | 1030 | 149 | 109 | 16.4 |
| DLD1 | 490 | 127 | 71.4 | 40.1 |
| CRO2B | 665 | 149 | 56.1 | 7.46 |
| PANC1 | 759 | 177 | 211 | 57.3 |
| SW620 | 980 | 134 | 54.9 | 13.4 |

Table 3 above shows that most of the tested compounds present antitumor activity with possible promising application. The compounds are sorted according to decreasing order of antitumor power observed by cytotoxicity tests:
9>>46>5>4

Compound 6 exhibited the highest antitumor activity in the series, although it also presented significant activity against fibroblasts, thus translating its nonspecific behavior.

On the other hand, compound 7 exhibited little antitumor activity when compared to the results of its analogues of the table.

It is important to emphasize that, in the vast majority, these compounds were not so selective for tumor cells, which was demonstrated by their activities before fibroblasts. However, both rationally directed molecular modifications and new formulations may lead to greater selectivity of these compounds for tumor cells and therefore, such compounds should not be disregarded in later studies.

### Colo205 Cell Tests

### Cell Count

The compounds were tested for their ability to reduce the number of cells in the tumor, compared to cells treated with culture medium only. In Table 4, the results obtained are shown.

TABLE 4
Inhibitory activity in the number of tumor cells line Colo205.

| | Relative number of cells |
|---|---|
| Compound | (% ± standard deviation) |
| Control | 100 ± 0.339 |
| 1 | 70.2 ± 0.339 |
| 27 | 32.3 ± 1.017 |
| 6 | 12.7 ± 0.339 |
| 2 | 12.7 ± 0.339 |

Data show that, with the exception of compound 1, all other compounds were able to significantly reduce the number of cells in the culture of the colon tumoral lineage Colo205.

Determination of Apoptotic Cells (subG1)-Colo205

In this experiment, the proportion of apoptotic cells was identified, that is, cells showing DNA content below normal (sub-diploid) in flow cytometry testing.

In Table 5, the results obtained are shown.

| Compound | Percentage of cells |
|---|---|
| Control | 5.1 ± 0.156 |
| 1 | 5.2 ± 0.566 |
| 27 | 18.0 ± 0.354 |
| 6 | 41.8 ± 0.141 |
| 2 | 35.7 ± 1.485 |

These data indicate that compounds 6 and 27, respectively, significantly induce cell death programmed by apoptosis in accordance with the values obtained by the minimum inhibitory concentration (CI50), shown above in Table 5.

### Analysis of the Cell Cycle Phases

The compounds of this family were also studied for the distribution of the population of tumor cells in the different phases of the cell cycle: quiescent cells, non-proliferating (G1/G0 phase), which present DNA/RNA synthesis capacity (S phase) and cell division cells (G2/M phase).

TABLE 6
Cell cycle phase

| | Cell cycle phase (%) | | |
|---|---|---|---|
| Compound | G1/G0 | G2M | S |
| Control | 62.3 ± 0.21 | 13.8 ± 2.33 | 23.9 ± 2.12 |
| 1 | 48.0 ± 2.54 | 5.0 ± 2.26 | 47.0 ± 0.28 |
| 27 | 54.2 ± 0.35 | 12.4 ± 0.63 | 33.4 ± 0.21 |
| 6 | 64.3 ± 1.69 | 16.0 ± 0.28 | 19.7 ± 1.48 |
| 2 | 90.0 ± 0.91 | 0.8 ± 0.42 | 9.15 ± 0.49 |

The compounds affect the cellular cycle differently, namely:
Phase S (synthesis)→1 and 27
Phase G1/G0 (quiescent cells)→2

Compound 6 presented higher cytotoxic activity among the tested compounds, however, it presented the lowest effects on cell distribution in the cell cycle and also significant inhibitory capacity of the S-phase cell population (DNA/RNA synthesis).

After the anti-proliferative results presented above on the prenylated compounds, it is concluded that, with the exception of compound 1, all other compounds in the series exhibited significant cytotoxic activity against a wide variety of tumor lines, showing good specificity.

The CI50 values of these compounds correlate significantly with the values of reduction in the number of total cells (correlation coefficient, r=0.995) to the number of apoptotic cells in the sub G1 phase (R=0.857) and to cell stoppage in phase S of the cell cycle (r=0.812), indicating that these compounds present antiproliferative activity, S-phase stoppage and apoptosis induction as some of the effects of the tested compounds.

The O-prenylated compound in the ortho position, 2-(3-methylbut-2-enyloxy)acetophenone, presented anti-proliferative activity much higher than that shown by compound 1 (replaced in para).

It can be concluded that compound 2 presented a higher antitumor activity than its raw material, compound 1.
2>1

The increase in electronic conjugation is supposed to lead to an increase in anti-proliferative action.

This information guides the conduct of studies on the chemical structure qualitative antiproliferative activity (SAR) in order to be possible to assess these facts more accurately and thus to find new prototypes with greater antitumor activity.

Other Results Obtained from the Push-Pull Butadienes Studied:

Compound 16: 4-(1-chloro-4-nitro-buta-1,3-dienyl)phenyl benzoate, exhibited cytotoxic activity in B16F10 melanoma cells. After 24 hours of culture in the presence and absence of the compound in the various concentrations, the straight equation and the linear regression curve were calculated in the Graph Pad Prism Instat program. This compound presented CI50% of 34.92 μg/mL or 106 □M and with high correlation or specificity (r2=0.93) in B16F10 melanoma cells.

Chart 3—Cytotoxic activity of compound 16: 4-(1-chloro-4-nitro-buta-1,3-dienyl)phenyl benzoate in murine melanoma cells B16 F10, determined by the MTT colorimetric test.

Compound 7: 2-(1-chloro-4-nitro-buta-1,3-dienyl)phenyl benzoate, exhibited cytotoxic activity in B16F10 melanoma cells. After 24 hours of culture in the presence and absence of the compound in the various concentrations, the straight equation and the linear regression curve were calculated in the Graph Pad Prism Instat program. This compound presented CI50% of 3.27 μg/mL or 9.93 □M and with high correlation or specificity (r2=0.94) in B16F10 melanoma cells.

Chart 4—Cytotoxic activity of compound 7: 2-(1-chloro-4-nitro-buta-1,3-dienyl)phenyl benzoate in murine melanoma cells B16 B16F10, determined by the MTT colorimetric test.

Compound 6: 2-(1-Chloro-4,4 dyciano-buta-1,3-dienyl)phenyl benzoate, exhibited cytotoxic activity in B16F10 melanoma cells. After 24 hours of culture in the presence and absence of the compound in the various concentrations, the straight equation and the linear regression curve were calculated in the Graph Pad Prism Instat program. This compound presented CI50% of 30.60 μg/mL or 91.6 □M and with high correlation or specificity (r2=0.98) in B16F10 melanoma cells.

Chart 5—Cytotoxic activity of compound 6: 2-(1-chloro-4,4-dicyano-buta-1,3-dienyl)phenyl benzoate in murine melanoma cells B16 F10, determined by the MTT colorimetric test.

### Comparison of Cytotoxic Activities of Organic Compounds in Melanoma Cells B16F10

| Cell Line/Synthetic Organic Compound | CI50% (μg/mL) |
|---|---|
| Melanoma B16F10 | |
| 14 | 168.42 |
| 16 | 34.92 |
| 7 | 3.27 |
| 6 | 30.60 |

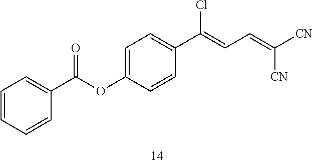 [SMILES: N#CC(C#N)=C/C=C(\Cl)c1ccc(OC(=O)c2ccccc2)cc1]

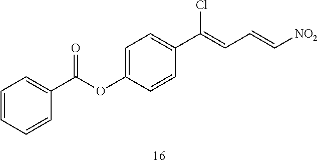 [SMILES: O=C(Oc1ccc(/C(Cl)=C/C=C/[N+](=O)[O-])cc1)c1ccccc1]

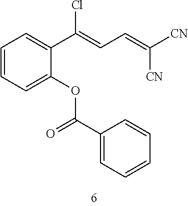 [SMILES: N#CC(C#N)=C/C=C(\Cl)c1ccccc1OC(=O)c1ccccc1]

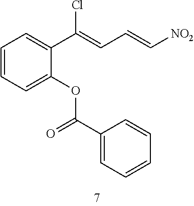 [SMILES: O=C(Oc1ccccc1/C(Cl)=C/C=C/[N+](=O)[O-])c1ccccc1]

Compound 6: 2-(1-chloro-4,4-dicyano-buta-1,3-dienyl)phenyl benzoate
Compound 7: 2-(1-chloro-4-nitro-buta-1,3-dienyl)phenyl benzoate
Compound 14: 4-(1-chloro-4,4-dicyano-buta-1,3-dienyl)phenyl benzoate
Compound 16: 4-(1-chloro-4-nitro-buta-1,3-dienyl)phenyl benzoate

Compounds with high cytotoxic and selective capacity to induce toxicity and inhibit tumor cell proliferation and not to attack normal cells were in ascending order: compound 7, compound 6 and compound 16. It should be noted that the butadiene push-pull compound 6 had been superimposed in CI50% by compound 7, which is a push-pull butadiene with replacement in the ortho position, as compound 6, but that instead of counting on two groups CN, contains a nitro group. This simple structural difference motivated the achievement of a compound 7 almost ten times more active than the compound 6 before this cell line. In this comparison, it is again observed that the compounds replaced in the ortho position present a significantly higher antitumor activity, that their isomers in position replaced in para position.
7>>16 and 6>>14

Seeking to find new push-pull butadienes with even better antitumor activity against the lineage (B316F10), the following compounds were synthesized:

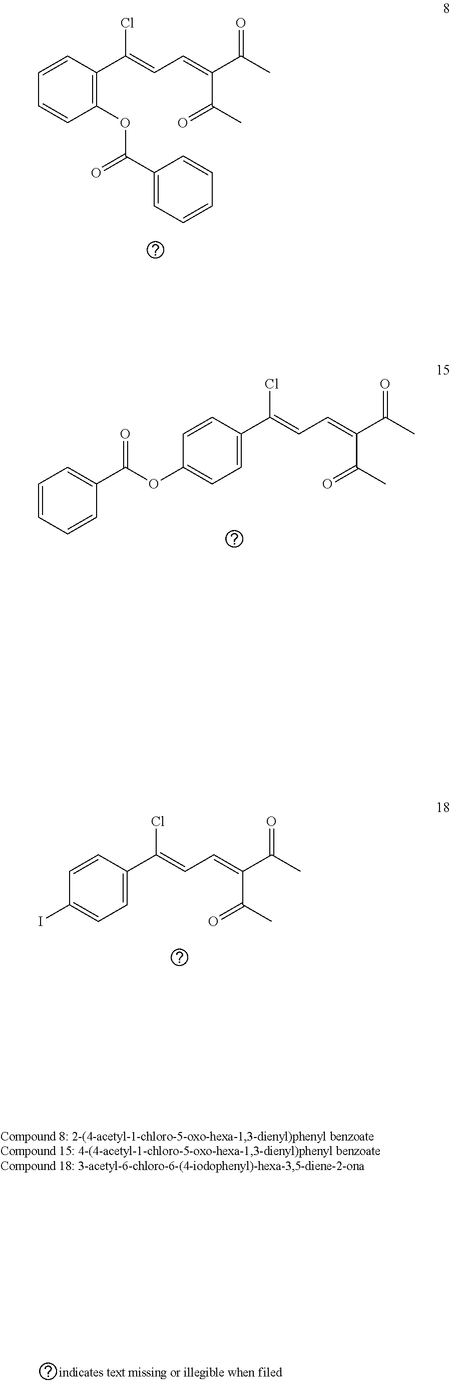 [SMILES: CC(=O)C(=C/C=C(\Cl)c1ccccc1OC(=O)c1ccccc1)C(C)=O ; CC(=O)C(=C/C=C(\Cl)c1ccc(OC(=O)c2ccccc2)cc1)C(C)=O ; CC(=O)C(=C/C=C(\Cl)c1ccc(I)cc1)C(C)=O]

In this case, acetylene acetone was selected as CH-acid compound for Knoevenagel condensation with the corresponding chlorovinylaldehyde. In this choice it was considered that in curcumin, this structural unit is present. It was observed in these derivatives that the isomer in ortho position continues to show the highest cytotoxicity, but the difference is not so marked. A new iodized push-pull butadiene of compound 18 was obtained: 3-acetyl-6-chloro-6-(4-iodinephenyl)-hexa-3,5-diene-2-ona, almost 7 times more active, being a non-phenolic compound.

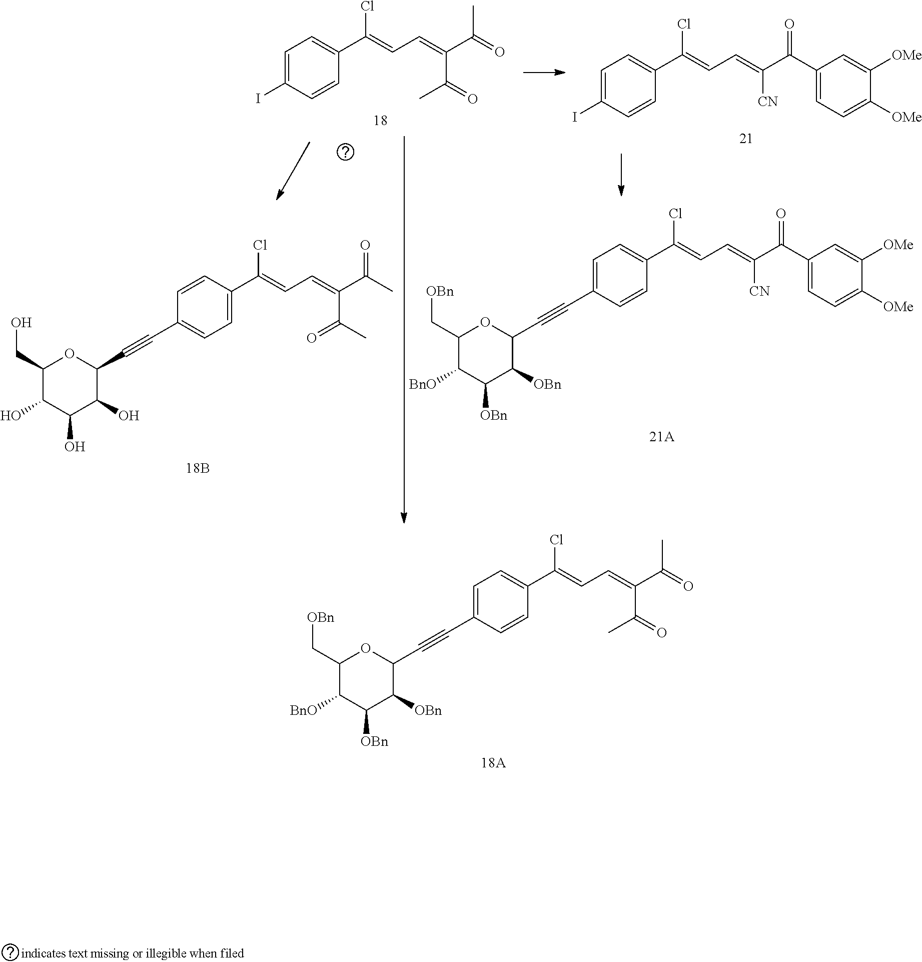

Taking into account this new fact, from 3-acetyl-6-chloro-6-(4-iodophenyl)-hexa-3,5-diene-2-ona new derivatives and their structural analogues with the objective of developing new more effective anti-tumor active principles.

| | CI50% tumor | | | | CI50% Normal |
|---|---|---|---|---|---|
| | cells in □M | | | | cells in □M |
| Compound | 4T1 | MCF-7 | SCC9 | SCC25 | Huvec |
| 18 | 3.715 | 0.6665 | 10.86 | — | — |
| 21 | 6.438 | 0.293 | 0.8581 | — | 16.43 |
| 21A | 2.698 | 0.5668 | 3.547 | — | 23.09 |
| 18A | 1.66 | 21.29 | 10.36 | 4.198 | — |
| 18B | 0.4306 | — | 0.4449 | 0.4528 | 0.2926 |

4T1 (murine mammary carcinoma);
MCF-7 (human mammary adenocarcinoma);
SCC9 (squamous-cell carcinoma of the oral cavity);
SCC25 (squamous-cell carcinoma of the oral cavity);
HUVEC (Normal human endothelial cell);
FN-1 (Normal human fibroblast)
Compound 18: 3-acetyl-6-chloro-6-(4-iodophenyl)-hexa-3,5-diene-2-ona
Compound 21: 5-chlorine-2-(3,4-dimethoxy-benzoyl)-5-(4-iodine-phenyl)-penta-2,4-dienonitrile
Compound 21A: 5-(chloro-2-(3,4-dimethoxybenzyl)-5-(4-ethynyl-2,3,4,6-benzil-β-D-galactopyranoside)-penta-2,4-dienonitrile
Compound 18A: 3-((Z)-3-(4-(2-((2S,3S,4R,5S)-3,4,5-tris(benzyloxy)-6-((benzyloxy)methyl)-tetrahydro-2H-piran-2-IL)ethinyl)phenyl)-3-chloroalilidene)pentane-2,4-dione
Compound 18B: 3-((Z)-3-chloro-3-(4-(2-((2S,3R,4R,5R)-tetrahydro-3,4,5-trihydroxy-6-(Hydroxymethy)-2H-pyran-2-il)ethynyl)phenyl)allilideno)pentano-2,4-dione

The derivative compounds 21, 21A and 18A presented excellent cytotoxicity against all tumor lines tested, highlighting compound 21 and compound 21A for showing good selectivity, mainly against human breast cancer. Compound 18B is undoubtedly the most active push-pull butadiene, but also the least selective one, perhaps due to the fact that this compound has all its free OH groups, which causes a greater adsorption on the cells, thereby increasing its cytotoxicity.

| | | | | | CI50% Normal | |
|---|---|---|---|---|---|---|
| Com- | CI50% tumor cells in □M | | | | cells in □M | |
| pound | 4TI | MCF-7 | SCC9 | SCC25 | HUVEC | FN-1 |
| 23 | 22.09 | 54.65 | 14.95 | 3.323 | 30.15 | 53.33 |
| 24 | — | — | — | — | 114.0 | 61.80 |
| 29 | 8.273 | 0.7005 | 9.22 | — | — | — |

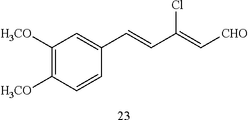 [SMILES: COc1ccc(/C=C/C(Cl)=C/C=O)cc1OC ; "23"]

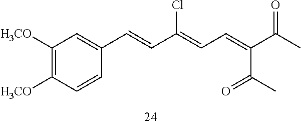 [SMILES: COc1ccc(/C=C/C(Cl)=C/C=C(C(C)=O)C(C)=O)cc1OC ; "24"]

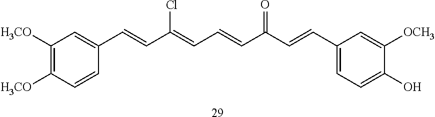 [SMILES: COc1cc(/C=C/C(=O)/C=C/C=C(Cl)/C=C/c2ccc(OC)c(OC)c2)ccc1O ; "29"]

HUVEC (Normal human endothelial cell);
FN-1 (Normal human fibroblast)
Compound 23: (4E)-3-chloro-5-(3,4-dimethoxyphenyl)penta-2,4-dienal
Compound 24: 3-((2Z,4E)-3-chloro-5-(3,4-dimethoxyphenyl)penta-2,4-
dien-ylidene)pentane-2,4-dione
Compound 29: (1E,4E,6Z, 8E)-7-chloro-1-(4-hydroxy-3-methoxyphenyl)-9-
(3,4-dimethoxyphenyl)nona-1,4,6,8-tetraen-3-ona

Regarding compounds 23, 24 and 29 were obtained by following the accumulated experience of inventors with regard to systems with high electronic conjugation with the same pattern of substitution of existing aromatic rings presented with excellent antitumor results derived from curcuminoids, demonstrated in other patents:

U.S. Pat. No. 7,432,401 of 7 Oct. 2008; Jose Agustin Quincoces Suerez and collaborators, Japan-Patent JP 5317290 of 19 Jul. 2013; Jose Agustin Quincoces Suerez and collaborators, European-Patent EP 2054365 of 29 Sep. 2014; Jose Agustin Quincoces Suerez and collaborators, U.S. Pat. No. 8,859,625 of 14 Oct. 2014; Jose Agustin Quincoces Suerez and collaborators, Japan-Patent JP; 5802658 of Nov. 11, 2015 of JP; Jose Agustin Quincoces of JP; of JP; Jose Agustin Quincoces Suerez and collaborators and U.S. Pat. No. 9,381,169 of 5 Jul. 2016; Jose Agustin Quincoces Suerez and collaborators.

These three push-pull systems also showed good antiproliferative activity.

In vitro antiproliferative studies were carried out against tumor strains, such as:

| Cell line | Origin |
|---|---|
| CaCo2 | Colon |
| HT-29 | Colon |
| Colo205 | Colon |
| SW620 | Colon |
| CRO2B | Carcinoid |
| MIAP2 | Pancreas |
| BxPC3 | Pancreas |
| PANC1 | Pancreas |
| B16F10 | Murine melanoma |
| 4T1 | Murine mammary carcinoma); |
| MCF-7 | Human mammary adenocarcinoma |
| SCC9 | Squamous-cell carcinoma of the oral cavity |
| SCC25 | Squamous-cell carcinoma of the oral cavity |
| NCI460 | Human lung cancer |
| NCIADR | Multiple drug resistant ovary |
| HUVEC | Normal human endothelial cell |
| FN-1 | Normal human fibroblast |

What is claimed is:

1) Process to prepare the formula compound 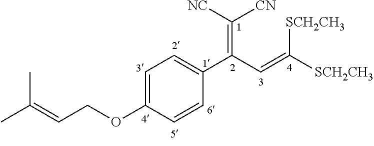
by means of push-pull butadienes characterized by the fact that it comprises a mixture consisting of 0.0068 mol 2-cyan-3-[4-(3-methylbut-2-enyloxy)-phenyl]-but-2-en-nitrile and 0.0068 mol carbon disulfide in 10 mL of absolute dimethylformamide, stir the room temperature under argon atmosphere, add 0.0136 mol of sodium hydride and stir under argon for eight hours, add 0.0136 mol of ethyl iodide (1097 μL) under agitation for 4 hours and pour the mixture into cold water.

2) Process, according to claim 1, characterized by the compound being extracted by solvent distilled with the help of the vacuum rotary evaporator, preferably ether, and the organic phase is washed three times with water and dried with anhydrous sodium sulphate.

3) (canceled)

4) Process, according to claim 1, characterized by the compound being purified using chromatographic column filled with silica gel using as eluent a mixture formed of toluene and ethyl acetate in the ratio 9/1.

5)-14) (canceled)

15) Process to prepare the formula compound 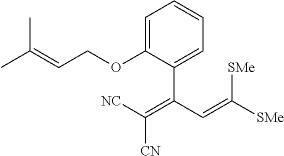 [SMILES: CSC(=CC(=C(C#N)C#N)c1ccccc1OCC=C(C)C)SC]
by means of push-pull butadienes characterized by the fact that it comprises a mixture consisting of 1.388 mmol 2-cyan-3-[2-(3-methylbut-2-enyloxy)-phenyl]-but-2-en-nitrile and 1.388 mmol of carbon disulfide in 5 mL of absolute dimethylformamide, stir the mixture at room temperature under argon atmosphere, added 0.002776 mol of hydride, stir under argon atmosphere for eight hours, and afterwards stir 2.77 mmol (173 μL) of ethyl iodide under agitation, the mixture being stirred for 4 hours and poured into cold water.

16) (canceled)

17) Process, according to claim 15, characterized by the compound being extracted by solvent, distilled with the help of the vacuum rotary evaporator, preferably chloroform, and the organic phase is washed three times with water and dried with anhydrous sodium sulphate.

18) (canceled)

19) Process, according to claim 15, characterized by the compound being purified using chromatographic column filled with silica gel, using as a mobile phase a mixture formed of n-heptane and ethyl acetate in the ratio 1/2.

20)-49) (canceled)

50) Compound, characterized by being 2-[2-(3-metilbut-2-eniloxi)-phenyl]-4,4-bis-methylsulfanyl-buta-1,3-dien-1,1-dicarbonitrile and having the structural formula

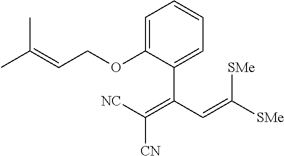 [SMILES: CSC(=CC(=C(C#N)C#N)c1ccccc1OCC=C(C)C)SC]

51)-59) (canceled)

60) Use of compounds, obtainable by processes as defined in claim 1, characterized by the fact that it is to prepare a pharmaceutical composition for cancer treatment selected from the group consisting of colon carcinoma, carcinoid tumor, lung cancer, pancreas cancer, murine melanoma, murine mammary carcinoma, human breast adenocarcinoma, oral cavity squamous cell carcinoma, multiple drugs resistant ovary.

61)-62) (canceled)

63) Use of compound obtainable by process as defined in claim 15, characterized by the fact that it is to prepare a pharmaceutical composition for cancer treatment selected from the group consisting of colon carcinoma, carcinoid tumor, lung cancer, pancreas cancer, murine melanoma, murine mammary carcinoma, human breast adenocarcinoma, oral cavity squamous cell carcinoma, multiple drugs resistant ovary.

64) Use of compound as defined in claim 50, characterized by the fact that it is for cancer treatment selected from the group consisting of colon carcinoma, carcinoid tumor, lung cancer, pancreas cancer, murine melanoma, murine mammary carcinoma, human breast adenocarcinoma, oral cavity squamous cell carcinoma, multiple drugs resistant ovary.
